(19) United States
(12) Patent Application Publication
(10) Pub. No.: US 2012/0295355 A1
(43) Pub. Date: Nov. 22, 2012

(54) NUCLEIC ACID DELIVERY USING MODIFIED CHITOSANS

(76) Inventors: Shenda Baker, Upland, CA (US); William P. Wiesmann, Washington, DC (US); Ruth Baxter, Los Angeles, CA (US); Snezna Rogelj, Socorro, NM (US)

(21) Appl. No.: 13/417,992

(22) Filed: Mar. 12, 2012

Related U.S. Application Data
(63) Continuation of application No. 13/146,990, now abandoned.
(60) Provisional application No. 61/148,338, filed on Jan. 29, 2009.

(51) Int. Cl. C08B 37/08; C12N 15/85; C12N 15/87

(52) U.S. Cl. 435/455; 536/20

(57) ABSTRACT

The present invention is directed to the delivery of nucleic acids in a non-viral vector to cells by positively charged chitosan derivatives, including but not limited to chitosan-arginine, chitosan-lysine and chitosan-histidine.

### PRIORITY CLAIM

The present application claims the benefit of U.S. Provisional Application No. 61/148,338, filed Jan. 29, 2009, the contents of which are not incorporated herein by reference.

### FIELD OF THE INVENTION

The invention relates to derivatized chitosan and its use as a carrier for nucleic acids, e.g., in the delivery of nucleic acids to cells.

### BACKGROUND

Chitosan has been widely used as a carrier for drugs, proteins and nucleic acids due to its nature as a biocompatible, non-toxic polysaccharide and its ability to be complexed, delivered in solution or precipitated with these deliverable agents. In particular, much interest has been focused on optimizing chitosan's use in non-viral delivery of DNA, RNA and a range of nucleic acid compositions due to the complexities and potential toxicity of the viral envelope. Furthermore, the preparation of chitosan complexes for such delivery and transfection has been described in the literature. (see for example J. Akbuga, Plasmid-DNA loaded chitosan microspheres for in vitro IL-2 expression, European J of Pharmaceutics and Biopharmaceutics 58 (2004), 501-507; H.-I. Mao, Chitosan-DNA nanoparticles as gene carriers: synthesis, characterization and transfection efficiency, J of Controlled Release 70 (2001) 399-421; K. Roy Oral gene delivery with chitosan-DNA nanoparticles generates immunologic protection in a murine model of peanut allergy, Nature 5(40) (1999) 387-391; T. Kiang The effect of the degree of chitosan deacetylation on the efficiency of gene transfection, Biomaterials 25 (204) 5293-5301; W. Liu An investigation on the physicochemical properties of chitosan/DNA polyelectrolyte complexes, Biomaterials 26(5) (2005) 2705-2711.)

### SUMMARY OF THE INVENTION

It is an objective of the present invention to provide a composition, complex, or particle comprising a chitosan derivative including but not limited to chitosan-arginine, chitosan-lysine and chitosan-histidine, and others, and a nucleic acid that provides efficient delivery to a cell membrane.

It is also an objective of the present invention to provide a composition, complex, or particle that comprises chitosan derivatives that are soluble at pH 7 including those having a molecular weight below 25 kDa, and methods of making such compositions, complexes and particles.

Also described herein are methods of transfecting cells comprising contacting the cells with a chitosan derivative and/or a nucleic acid.

In one aspect, the invention features a method of transfecting a cell with a nucleic acid comprising: providing a cell; and contacting said cell with a composition comprising said nucleic acid, and a functionalized chitosan of the following formula (I):

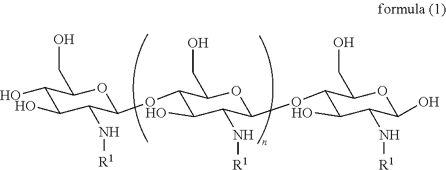

wherein:

n is an integer between 20 and 6000; and

each R¹ is independently selected for each occurrence from hydrogen, acetyl, and either:

a) a group of formula (II):

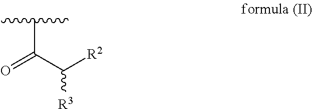

wherein R² is hydrogen or amino; and

R³ is amino, guanidino, C₁-C₆ alkyl substituted with an amino or guanidino moiety, or a natural or unnatural amino acid side chain;

or

b) R¹, when taken together with the nitrogen to which it is attached, forms a guanidine moiety;

wherein at least 25% of R¹ substituents are H, at least 1% of R¹ substituents are acetyl, and at least 2% of R¹ substituents are a group of formula (II) or are taken together with the nitrogen to which they are attached to form a guanidine moiety.

In some embodiments, the composition comprises a complex, wherein the complex comprises a chitosan derivative and a nucleic acid. In some embodiments, the complex is nanometers in dimension, for example, due to the nature of the molecules involved, e.g. the chitosan derivative and/or the nucleic acid. In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In one aspect, the invention features a method of transfecting a cell with a nucleic acid comprising:

providing a cell; and

contacting said cell with a composition comprising said nucleic acid, and a functionalized chitosan of the following formula (I) wherein at least 90% by number or weight of R¹ moieties are as defined in formula (I) (e.g., at least about 95%, at least about 96%, at least about 97%, at least about 98%, or at least about 99%):

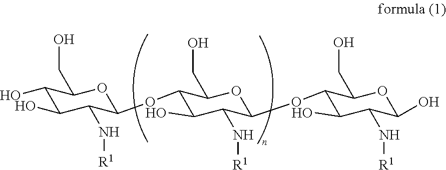

wherein:

n is an integer between 20 and 6000; and

each R¹ is independently selected for each occurrence from hydrogen, acetyl, and either:

a) a group of formula (II):

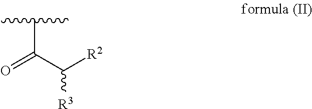

wherein R² is hydrogen or amino; and

R³ is amino, guanidino, C₁-C₆ alkyl substituted with an amino or guanidino moiety, or a natural or unnatural amino acid side chain;

or

b) R¹, when taken together with the nitrogen to which it is attached, forms a guanidine moiety;

wherein at least 25% of R¹ substituents are H, at least 1% of R¹ substituents are acetyl, and at least 2% of R¹ substituents are a group of formula (II) or are taken together with the nitrogen to which they are attached to form a guanidine moiety.

In some embodiments, the composition comprises a complex, wherein the complex comprises a chitosan derivative and a nucleic acid. In some embodiments, the complex is nanometers in dimension, for example, due to the nature of the molecules involved, e.g. the chitosan derivative and/or the nucleic acid. In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In some embodiments, between 25-95% of R¹ substituents are hydrogen.

In some embodiments, between 55-90% of R¹ substituents are hydrogen.

In some embodiments, between 1-50% of R¹ substituents are acetyl.

In some embodiments, between 4-20% of R¹ substituents are acetyl.

In some embodiments, between 2-50% of R¹ substituents are a group of formula (II).

In some embodiments, between 4-30% of R¹ substituents are a group of formula (II).

In some embodiments, 55-90% of R¹ substituents are hydrogen, 4-20% of R¹ substituents are acetyl, 4-30% of R¹ substituents are a group of formula (II).

In some embodiments, R² is amino and R³ is an arginine side chain.

In some embodiments, R¹ is selected from one of the following:

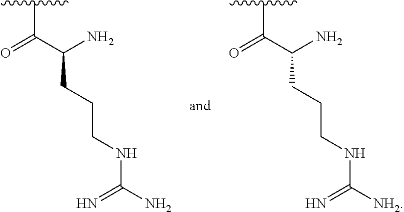

In some embodiments, R² is amino and R³ is a lysine side chain.

In some embodiments, R¹ is selected from one of the following:

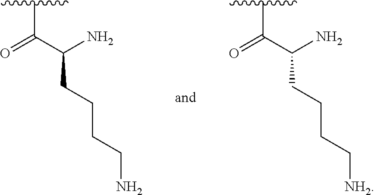

In some embodiments, R² is amino and R³ is a histidine side chain.

In some embodiments, R¹ is selected from one of the following:

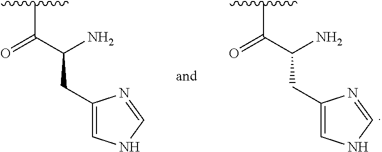

In some embodiments, at least 1% of R¹ substituents are selected from one of the following:

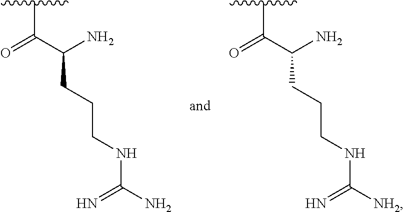

AND at least 1% of R¹ substituents are selected from the following:

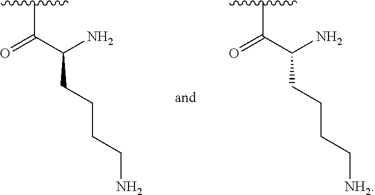

In some embodiments, R² is amino and R³ is a substituted C₁-C₆ alkyl.

In some embodiments, R³ is C₁-C₆ alkyl substituted with an amino group.

In some embodiments, R³ is C₁ alkyl substituted with an amino group.

In some embodiments, R³ is C₂ alkyl substituted with an amino group.

In some embodiments, R³ is C₃ alkyl substituted with an amino group.

In some embodiments, R¹ is selected from one of the following:

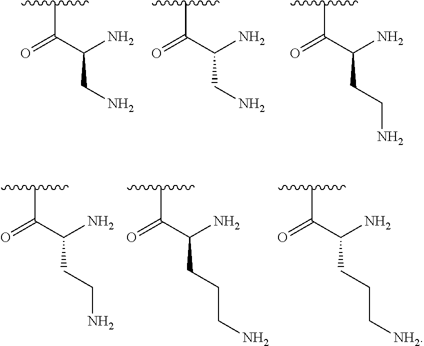

In some embodiments, R³ is C₁-C₆ alkyl substituted with a guanidino group.

In some embodiments, R³ is C₁ alkyl substituted with a guanidino group.

In some embodiments, R³ is C₂ alkyl substituted with a guanidino group.

In some embodiments, R¹ is selected from one of the following:

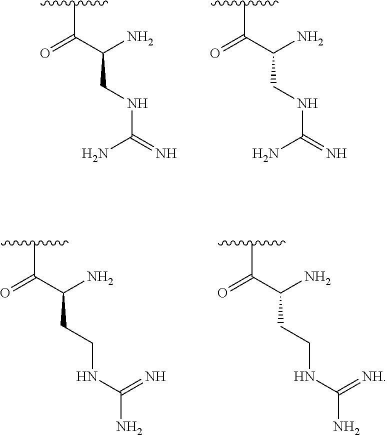

In some embodiments, wherein R² is amino that is substituted with a nitrogen protecting group prior to substitution on chitosan and removed subsequent to substitution on chitosan.

In some embodiments, the nitrogen protecting group is tert-butyloxycarbonyl (Boc).

In some embodiments, in the synthetic process a nitrogen protecting group is used, which can provide an intermediate polymer having a nitrogen protecting group such as Boc.

In some embodiments, R² is amino.

In some embodiments, R² is hydrogen and R³ is amino.

In some embodiments, R² is hydrogen and R³ is guanidino.

In some embodiments, R² is hydrogen and R³ is a substituted C₁-C₆ alkyl.

In some embodiments, R³ is C₁-C₆ alkyl substituted with an amino group.

In some embodiments, R³ is C₁ alkyl substituted with an amino group.

In some embodiments, R³ is C₂ alkyl substituted with an amino group.

In some embodiments, R³ is C₃ alkyl substituted with an amino group.

In some embodiments, R³ is C₄ alkyl substituted with an amino group.

In some embodiments, R³ is C₅ alkyl substituted with an amino group.

In some embodiments, R³ is selected from one of the following:

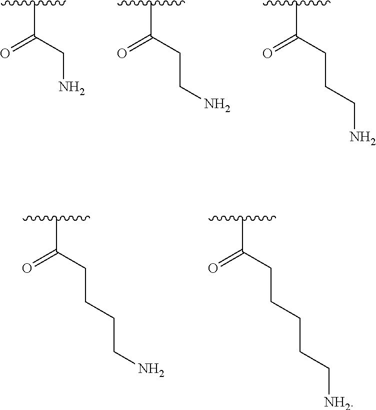

In some embodiments, R³ is C₁-C₆ alkyl substituted with a guanidino group.

In some embodiments, R³ is C₁ alkyl substituted with a guanidino group.

In some embodiments, R³ is C₂ alkyl substituted with a guanidino group.

In some embodiments, R³ is C₃ alkyl substituted with a guanidino group.

In some embodiments, R³ is C₄ alkyl substituted with a guanidino group.

In some embodiments, R³ is C₅ alkyl substituted with a guanidino group.

In some embodiments, R¹ is selected from one of the following:

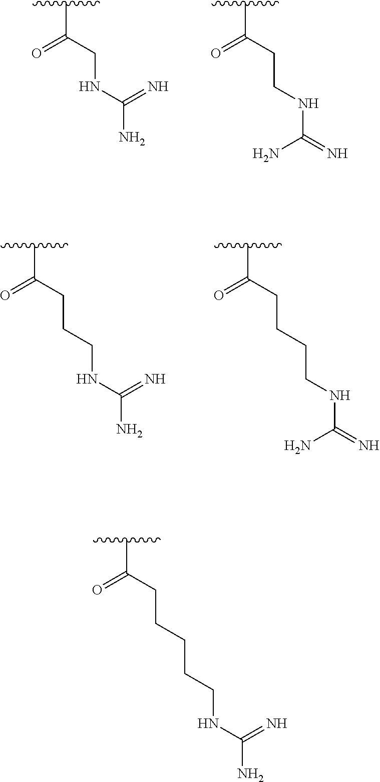

In some embodiments, at least 25% of IV substituents are H, at least 1% of IV substituents are acetyl, and at least 2% of R¹ substituents independently selected from any of the formulae specifically shown above.

In some embodiments, the functionalized chitosan of formula (I) may be further derivatized on the free hydroxyl moieties.

In some embodiments, the molecular weight of the functionalized chitosan is between 5,000 and 1,000,000 Da.

In some embodiments, the molecular weight of the functionalized chitosan is between 5,000 and 350,000 Da.

In some embodiments, the molecular weight of the functionalized chitosan is between 5,000 and 60,000 Da.

In some embodiments, the molecular weight of the functionalized chitosan is between 5,000 and 60,000 Da.

In some embodiments, the molecular weight of the functionalized chitosan is between 5,000 and 45,000 Da.

In some embodiments, the molecular weight of the functionalized chitosan is between 5,000 and 35,000 Da.

In some embodiments, the molecular weight of the functionalized chitosan is between 5,000 and 25,000 Da.

In some embodiments, the functionalized chitosan is soluble in aqueous solution between pH 6 and 8.

In some embodiments, the functionalized chitosan is soluble in aqueous solution between pH 6.8 and pH 7.4.

In some embodiments, the functionalized chitosan is substantially free of other impurities.

In some embodiments, said composition further comprises a lipid, e.g., a cationic, anionic or neutral lipid (for example, as used in a transfection agent).

In some embodiments, functionalized chitosan of formula (I) and lipid are present in a ratio of about 0.001 to 1, 0.005 to 1, 0.01 to 1, 0.05 to 1, 0.1 to 1, 0.5 to 1, 1 to 1, 5 to 1, 10 to 1, 50 to 1, 100 to 1, 500 to 1, or 1000 to 1, on a wt/wt basis.

In some embodiments, the nucleic acid and lipid are present in a ratio of about 0.001 to 1, 0.005 to 1, 0.01 to 1, 0.05 to 1, 0.1 to 1, 0.5 to 1, 1 to 1, 5 to 1, 10 to 1, 50 to 1, 100 to 1, 500 to 1, or 1000 to 1, on a wt/wt basis.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, an antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In some embodiments, the mass ratio of the nucleic acid (e.g., DNA) to the derivatized chitosan (e.g., chitosan-arginine) when one or more lipids or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (n) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In one aspect, the invention features a pharmaceutical composition comprising a nucleic acid and a functionalized chitosan as described herein (e.g., a chitosan of formula (I) or a functionalized chitosan wherein at least 90% by number or weight of R¹ moieties of the functionalized chitosan are as defined as in formula (I) (e.g., at least about 95%, at least about 96%, at least about 97%, at least about 98%, or at least about 99%)). In some embodiments, the pharmaceutical composition can be administered to transfect a cell with said nucleic acid.

In some embodiments, the composition comprises a complex, wherein the complex comprises a chitosan derivative and a nucleic acid. In some embodiments, the complex is nanometers in dimension, for example, due to the nature of the molecules involved, e.g. the chitosan derivative and/or the nucleic acid. In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In some embodiments, the composition further comprises a transfection reagent, e.g., a lipid, e.g., a cationic, anionic or neutral lipid.

In some embodiments, the composition comprises a plurality of functionalized chitosans of formula (I).

In some embodiments, the composition consists essentially of a plurality of functionalized chitosans of formula (I).

In some embodiments, the mean molecular weight of the functionalized chitosans is between 5,000 and 1,000,000 Da.

In some embodiments, the mean molecular weight of the functionalized chitosans is between 5,000 and 350,000 Da.

In some embodiments, the mean molecular weight of the functionalized chitosans is between 5,000 and 60,000 Da.

In some embodiments, the mean molecular weight of the functionalized chitosans is between 5,000 and 45,000 Da.

In some embodiments, the mean molecular weight of the functionalized chitosans is between 5,000 and 35,000 Da.

In some embodiments, the mean molecular weight of the functionalized chitosans is between 5,000 and 25,000 Da.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, an antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In another aspect, the invention features a kit comprising a nucleic acid and a functionalized chitosan of formula (I) to transfect a cell with said nucleic acid. In some embodiments, the kit also includes a lipid (e.g., a cationic, neutral, or anionic lipid) or formulation of lipids.

In some embodiments, the kit further comprises a nucleic acid comprising a reporter gene, e.g., a GFP.

In some embodiments, the chitosan is functionalized at between about 5% to about 40%, about 10% to about 35%, about 15% to about 30%, or about 20% to about 25%. In a preferred embodiment, the chitosan is functionalized at between about 15% to about 30%. For example, the chitosan can be functionalized at about 24, 25 or 26%.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, an antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In one aspect, the invention features a reaction mixture comprising a nucleic acid and a functionalized chitosan of formula (I), suitable, e.g., for transfection of the nucleic acid to a cell. In some embodiments, the reaction mixture also includes a lipid (e.g., a cationic, neutral, or anionic lipid) or a formulation of lipids.

In one aspect, the invention features a reaction mixture comprising a nucleic acid and a functionalized chitosan of formula (I), wherein at least 90% by number or weight of R¹ moieties of the functionalized chitosan are as defined as in formula (I) (e.g., at least about 95%, at least about 96%, at least about 97%, at least about 98%, or at least about 99%), suitable, e.g., for transfection of the nucleic acid to a cell. In some embodiments, the reaction mixture also includes a lipid (e.g., a cationic, neutral, or anionic lipid) or a formulation of lipids.

In some embodiments, the molecular weight of the functionalized chitosan is from about 5 kDa to about 1000 kDa, from about 5 kDa to about 350 kDa, from about 5 kDa to about 60 kDa, from about 5 kDa to about 45 kDa, from about 5 kDa to about 35 kDa, or from about 5 kDa to about 25 kDa. In some embodiments, the molecular weight of the functionalized chitosan is from about 10 to about 80 kDa. For example, the molecular weight of the functionalized chitosan can be 18, 35, 41, 57 or 70 kDa.

In some embodiments, the chitosan is functionalized at between about 5% to about 40%, about 10% to about 35%, about 15% to about 30%, or about 20% to about 25%. In a preferred embodiment, the chitosan is functionalized at between about 15% to about 30%. For example, the chitosan can be functionalized at about 24, 25 or 26%.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, an antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In some embodiments, the mass ratio of the nucleic acid (e.g., DNA) to the derivatized chitosan (e.g., chitosan-arginine) when lipid or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (μg) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In another aspect, the invention features a cell or population of cells produced by a method described herein.

In yet another aspect, the invention features a chitosan derivative complex comprising a functionalized chitosan of formula (I). In some embodiments, the complex includes at least one additional component such as a nucleic acid, a lipid (e.g., a cationic, neutral, or anionic lipid), a lipid formulation and/or a surfactant.

In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In some embodiments, the chitosan derivative complex further comprises a nucleic acid.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, an antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In some embodiments, the chitosan derivative complex further comprises a coprecipitate. In some embodiments, the coprecipitate is a nucleic acid, a lipid, a formulation of lipids and/or a surfactant.

In some embodiments, the mass ratio of the nucleic acid (e.g., DNA) to the derivatized chitosan (e.g., chitosan-arginine) when lipid for lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (μg) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In yet another aspect, the invention features a chitosan derivative complex comprising a functionalized chitosan of formula (I), wherein at least 90% by number or weight of R¹ moieties on the functionalized chitosan are as defined as in formula (I) (e.g., at least about 95%, at least about 96%, at least about 97%, at least about 98%, or at least about 99%). In some embodiments, the complex includes at least one additional component such as a nucleic acid, a lipid, a lipid formulation and/or a surfactant.

In another aspect, the invention features a kit comprising: the chitosan derivative complex described herein; and instructions for use to transfect a nucleic acid to a cell. In some embodiments, the kit also includes a lipid (e.g., a cationic, neutral, or anionic lipid).

In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In some embodiments, the molecular weight of the functionalized chitosan is from about 5 kDa to about 1000 kDa, from about 5 kDa to about 350 kDa, from about 5 kDa to about 60 kDa, from about 5 kDa to about 45 kDa, from about 5 kDa to about 35 kDa, or from about 5 kDa to about 25 kDa. In some embodiments, the molecular weight of the functionalized chitosan is from about 10 to about 80 kDa. For example, the molecular weight of the functionalized chitosan can be 18, 35, 41, 57 or 70 kDa.

In some embodiments, the chitosan is functionalized at between about 5% to about 40%, about 10% to about 35%, about 15% to about 30%, or about 20% to about 25%. In a preferred embodiment, the chitosan is functionalized at between about 15% to about 30%. For example, the chitosan can be functionalized at about 24, 25 or 26%.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, a antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In some embodiments, the mass ratio of the nucleic acid (e.g., DNA) to the derivatized chitosan (e.g., chitosan-arginine) when lipid or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (μg) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In yet another aspect, the invention features a chitosan derivative/nucleic acid complex, wherein the complex comprises: a functionalized chitosan of formula (I); and a nucleic acid. In some embodiments, the complex includes at least one additional component such as a nucleic acid, a lipid, a lipid formulation and/or a surfactant.

In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In yet another aspect, the invention features a chitosan derivative/nucleic acid complex, wherein the complex comprises: a functionalized chitosan of formula (I), at least 90% by number or weight of R¹ moieties of the functionalized chitosan are as defined as in formula (I) (e.g., at least about 95%, at least about 96%, at least about 97%, at least about 98%, or at least about 99%); and a nucleic acid. In some embodiments, the complex includes at least one additional component such as a nucleic acid, a lipid, and/or a surfactant.

In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, an antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In some embodiments, the mass ratio of the nucleic acid (e.g., DNA) to the derivatized chitosan (e.g., chitosan-arginine) when lipid or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (pig) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In one aspect, the invention features a method of making a chitosan derivative/nucleic acid complex comprising a functionalized chitosan of formula (I), the method comprising: providing a functionalized chitosan of formula (I); providing a nucleic acid; contacting the functionalized chitosan and the nucleic acid, thereby making a chitosan derivative/nucleic acid complex. In some embodiments, the complex includes at least one additional component such as a nucleic acid, a lipid, a lipid formulation and/or a surfactant.

In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In one aspect, the invention features a method of making a chitosan derivative/nucleic acid complex comprising a functionalized chitosan of formula (I), the method comprising: providing a functionalized chitosan of formula (I), wherein at least 90% by number or weight of R¹ moieties of the functionalized chitosan are as defined as in formula (I) (e.g., at least about 95%, at least about 96%, at least about 97%, at least about 98%, or at least about 99%); providing a nucleic acid; contacting the functionalized chitosan and the nucleic acid, thereby making a chitosan derivative/nucleic acid complex. In some embodiments, the complex includes at least one additional component such as a nucleic acid, a lipid, and/or a surfactant.

In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In some embodiments, the method further comprises contacting the functionalized chitosan and/or the nucleic acid with a lipid or formulation of lipids.

In some embodiments, the functionalized chitosan and the nucleic acid are contacted (e.g., mixed) in water (e.g., without lipid), e.g., for less than 10 seconds, 20 seconds, 30 seconds, 1 minute, 2 minutes, 5 minutes, 10 minutes, 20 minutes, or 30 minutes.

In some embodiments, the contacting (e.g., mixing) results in a complex that is nanometers in dimension.

In some embodiments, the complex comprises a particle.

In some embodiments, the particle is nanometers in dimension.

In some embodiments, the functionalized chitosan and the nucleic acid are contacted (e.g., mixed) in a medium (e.g., a serum-free medium), e.g., for less than 10 seconds, 20 seconds, 30 seconds, 1 minute, 2 minutes, 5 minutes, 10 minutes, 20 minutes, or 30 minutes.

In some embodiments, the functionalized chitosan and the nucleic acid are contacted (e.g., mixed) in the absence of a lipid.

In some embodiments, the functionalized chitosan and the nucleic acid are contacted (e.g., mixed) in the presence of a lipid or lipid formulation.

In some embodiments, the method further comprises contacting the resulting complex with a cell.

In some embodiments, the molecular weight of the functionalized chitosan is from about 5 kDa to about 1000 kDa, from about 5 kDa to about 350 kDa, from about 5 kDa to about 60 kDa, from about 5 kDa to about 45 kDa, from about 5 kDa to about 35 kDa, or from about 5 kDa to about 25 kDa. In some embodiments, the molecular weight of the functionalized chitosan is from about 10 to about 80 kDa. For example, the molecular weight of the functionalized chitosan can be 18, 35, 41, 57 or 70 kDa.

In some embodiments, the chitosan is functionalized at between about 5% to about 40%, about 10% to about 35%, about 15% to about 30%, or about 20% to about 25%. In a preferred embodiment, the chitosan is functionalized at between about 15% to about 30%. For example, the chitosan can be functionalized at about 24, 25 or 26%.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, a antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In some embodiments, the mass ratio of the nucleic acid (e.g., DNA) to the derivatized chitosan (e.g., chitosan-arginine) when lipid or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (μg) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In one aspect, the invention features a method of delivering a nucleic acid to a cell comprising: providing chitosan derivative/nucleic acid complex comprising a functionalized chitosan of formula (I) and a nucleic acid; and contacting said complex with said cell, thereby delivering a nucleic acid to a cell.

In some embodiments, the complex comprises a particle, wherein the particle comprises a chitosan derivative and a nucleic acid. In some embodiments, the particle is nanometer in dimension, for example, due to the nature of the molecules involved, e.g. the chitosan derivative and/or the nucleic acid.

In one aspect, the invention features a method of delivering a nucleic acid to a cell comprising: providing chitosan derivative/nucleic acid complex comprising a functionalized chitosan of formula (I), wherein at least 90% by number or weight of R¹ moieties of the functionalized chitosan are as defined as in formula (I) (e.g., at least about 95%, at least about 96%, at least about 97%, at least about 98%, or at least about 99%), and a nucleic acid; and contacting said complex with said cell, thereby delivering a nucleic acid to a cell. In some embodiments, the complex includes at least one additional component such as a nucleic acid, a lipid, and/or a surfactant.

In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid.

In some embodiments, the nucleic acid has a molecular weight of about 5, 10, 50, 100, 250, 500, 750, 1000, or greater kD.

In some embodiments, the nucleic acid comprises a DNA or RNA.

In some embodiments, the nucleic acid is double stranded or single stranded.

In some embodiments, the DNA comprises a cDNA, an in vitro polymerized DNA, a plasmid DNA, a part of a plasmid DNA, a genetic material derived from a virus, a linear DNA, an expression cassette, a chimeric sequence, a recombinant DNA, a chromosomal DNA, an oligonucleotide, an anti-sense DNA, or a derivative thereof.

In some embodiments, the RNA comprises an oligonucleotide RNA, a tRNA (transfer RNA), an snRNA (small nuclear RNA), an rRNA (ribosomal RNA), an mRNA (messenger RNA), an in vitro polymerized RNA, a recombinant RNA, a chimeric sequences, an anti-sense RNA, an siRNA (small interfering RNA), an shRNA (small hairpin RNA), a miRNA (microRNA), a piRNA (Piwi-interacting RNA), a long non-coding RNA, an RNA derived from a virus, a ribozymes, or a derivative thereof.

In some embodiments, the nucleic acid comprises a therapeutic gene, e.g., a tumor suppressor gene, a antigenic gene, a cytotoxic gene, a cytostatic gene, a pro-drug activating gene, an apoptotic gene, a pharmaceutical gene, or an anti-angiogenesis gene.

In some embodiments, the nucleic acid comprises a nucleic acid sequence that promotes integration of the nucleic acid into the host genome, e.g., a Long Terminal Repeat (LTR).

In some embodiments, the nucleic acid comprises a vector.

In some embodiments, the vector comprises one or more of an origin of replication, a multicloning site, a selectable marker (e.g., an antibiotic resistance marker, or a β-galactosidase sequence), a promoter (e.g., a CMV promoter, or an inducible promoter), a polyadenylation signal, a Kozak sequence, an enhancer, an epitope tag (e.g., HA, myc, or GFP), a localization signal sequence, an internal ribosome entry sites (IRES), or a splicing signal.

In some embodiments, the mass ratio of the nucleic acid (e.g., DNA) to the derivatized chitosan (e.g., chitosan-arginine) when lipid or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (μg) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In some embodiments, the composition and methods described herein, can result in the sensitization of a cell line, for example, allowing a cell to be more efficiently transfected, which cell under other conditions may have poor transfection efficiency. A derivatized chitosan can result in a sensitization of a cell line, e.g., a cell line that is normally difficult to transfect. Exemplary cells are those in which transfection of cells without the derivatized chitosan is typically less than about 1/10,000, about 1/1,000, about 1/100, about 1/10, about ⅕, or about ½ of the transfection of cells in the presence of chitosan derivative. In some embodiments the derivatized chitosan results in an increase in transfection efficiency of at least about 25% (e.g., at least about 50%, at least about 100%, at least about 200%, at least about 400%, at least about 600%, at least about 800%, at least about 1000%, at least about 2000%, or at least about 4000%), e.g., as compared with a standard, e.g., the cells without treatment with derivatized chitosan.

In some embodiments, a combination of a derivatized chitosan and a transfection reagent (e.g., a lipid or lipofectamine 2000) can result in a sensitization of a cell line, e.g., a cell line that is normally difficult to transfect. Exemplary cells are those in which transfection of cells without the derivatized chitosan is typically less than about 1/10,000, about 1/1,000, about 1/100, about 1/10, about ⅕, or about ½ of the transfection of cells in the presence of chitosan derivative. In some embodiments the derivatized chitosan results in an increase in transfection efficiency of at least about 25% (e.g., at least about 50%, at least about 100%, at least about 200%, at least about 400%, at least about 600%, at least about 800%, at least about 1000%, at least about 2000%, or at least about 4000%), e.g., as compared with a standard, e.g., the cells without treatment with derivatized chitosan.

In one aspect, the invention features a method of transfecting a cell with a nucleic acid comprising: providing a cell; contacting said cell with a functionalized chitosan of formula (I); and contacting said cell with a nucleic acid, thereby transfecting the nucleic acid to the cell.

In some embodiments, the cell is contacted with the nucleic acid, e.g., less than 10 seconds, 20 seconds, 30 seconds, 1 minute, 2 minutes, 5 minutes, 10 minutes, 20 minutes, or 30 minutes, before it is contacted with the functionalized chitosan.

In some embodiments, the cell is contacted with the functionalized chitosan, e.g., less than 10 seconds, 20 seconds, 30 seconds, 1 minute, 2 minutes, 5 minutes, 10 minutes, 20 minutes, or 30 minutes, before it is contacted with the nucleic acid.

In some embodiments, the method further comprises contacting the cell with a lipid or lipid formulation.

In some embodiments, the mass ratio of the nucleic acid (e.g., DNA) to the derivatized chitosan (e.g., chitosan-arginine) when lipid or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (μg) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

In another aspect, the invention features a method of transfecting a cell with a nucleic acid comprising: providing a cell; contacting said cell with a functionalized chitosan of formula (I), wherein at least 90% by number or weight of R¹ moieties of the functionalized chitosan are as defined as in formula (I) (e.g., at least about 95%, at least about 96%, at least about 97%, at least about 98%, or at least about 99%); and contacting said cell with a nucleic acid, thereby transfecting the nucleic acid to the cell.

In some embodiments, the said cell is contacted with the nucleic acid, e.g., less than 10 seconds, 20 seconds, 30 seconds, 1 minute, 2 minutes, 5 minutes, 10 minutes, 20 minutes, or 30 minutes, before it is contacted with the functionalized chitosan.

In some embodiments, the cell is contacted with the functionalized chitosan, e.g., less than 10 seconds, 20 seconds, 30 seconds, 1 minute, 2 minutes, 5 minutes, 10 minutes, 20 minutes, or 30 minutes, before it is contacted with the nucleic acid.

In some embodiments, the method further comprises contacting the cell with a lipid or lipid formulation.

In some embodiments, the mass ratio of the nucleic acid (e.g., DNA) to the derivatized chitosan (e.g., chitosan-arginine) when lipid or lipid formulation (e.g., Lipofectamine 2000) is not present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 25, about 1 to 50, or about 1 to 100.

In some embodiments, the mass ratio of the nucleic acid (e.g., DNA) to the derivatized chitosan (e.g., chitosan-arginine) when one or more lipids or lipid formulation (e.g., Lipofectamine 2000) is present is about 1 to 0.05, about 1 to 0.1, about 1 to 0.25, about 1 to 0.5, about 1 to 1, about 1 to 1.25, about 1 to 2.5, about 1 to 5, about 1 to 10, about 1 to 15, about 1 to 20, about 1 to 25, about 1 to 50, about 1 to 75, about 1 to 100, about 1 to 200, or about 1 to 500. In a preferred embodiment, the ratio is about 1 to 5, about 1 to 10, or about 1 to 25.

In some embodiments, the mass:volume ratio of the derivatized chitosan (e.g., chitosan-arginine) (μg) to a lipid or lipid formulation (μL) is about 1 to 0.0025, about 1 to 0.005, about 1 to 0.01, about 1 to 0.025, about 1 to 0.05, about 1 to 0.1, about 1 to 0.25, about 1 to 0.5, about 1 to 2, about 1 to 10, about 1 to 20, about 1 to 50, about 1 to 100, or about 1 to 200. In a preferred embodiment, the ratio is about 1 to 0.25, or about 1 to 0.5.

### BRIEF DESCRIPTION OF THE DRAWINGS

FIG. 1A depicts the binding of chitosan-arginine to DNA in solution. Chitosan mass/DNA mass ratio=1.6.

FIG. 1B depicts the binding of chitosan-arginine to DNA in solution. Chitosan mass/DNA mass ratios=0.8 and 1.6.

FIG. 1C depicts the binding of chitosan-arginine to DNA in solution. Chitosan mass/DNA mass ratios=0.16, 0.8 and 1.6.

FIG. 2A depicts an example of the β-galactosidase activity measured by the color change in o-nitrophenyl-beta-D-galactopyranoside (ONPG) at 420 nm, in HeLa cells transfected by a chitosan derivative.

FIG. 2B depicts an example of the β-galactosidase activity measured by the color change in o-nitrophenyl-beta-D-galactopyranoside (ONPG) at 420 nm, in B-7 cells transfected by a chitosan derivative.

FIG. 3A depicts an example of the luciferase activity, measured in relative light units, in HEK 293T cells transfected by chitosan derivatives.

FIG. 3B depicts an example of the luciferase activity in NIH3T3 cells transfected by chitosan derivatives.

FIG. 4 depicts an example of the luciferase activity in NIH3T3 cells transfected by chitosan derivative with mass ratios of CA to DNA of 0.25, 1.25, 5, 10, 25, 50 and 100.

FIG. 5 depicts an example of the luciferase activity in NIH3T3 cells where the DNA and CA were either preincubated for 20 mins before addition, or added separately to the culture medium in either order of CA first followed by CA within 1 minute, or DNA first followed by CA within 1 minute.

FIG. 6A depicts an example of the luciferase activity in HEK 293T cells transfected by a combination of DNA plus chitosan derivative, and DNA plus chitosan derivative plus Lipofectamine 2000.

FIG. 6B depicts another example of the luciferase activity in NIH3T3 cells transfected by a combination of DNA plus chitosan derivative, and DNA plus chitosan derivative plus Lipofectamine 2000.

FIG. 7 depicts an example of the luciferase activity in NIH3T3 cells transfected by DNA plus chitosan derivative, and DNA plus chitosan derivative plus Lipofectamine 2000 with mass ratios of CA to DNA of 0.25, 1.25, 5, 10, 25, 50 and 100.

FIG. 8A depicts another example of the luciferase activity in HEK293T cells transfected by DNA plus chitosan derivatives alone or DNA plus CA plus Lipofectamine 2000. The graph is normalized to the lipofectamine 2000 only control.

FIG. 8B depicts another example of the luciferase activity in NIH3T3 cells transfected by DNA plus chitosan derivatives alone or DNA plus CA plus Lipofectamine 2000. The graph is normalized to the Lipofectamine 2000 only control.

FIG. 8C depicts another example of the luciferase activity in A549 cells transfected by DNA plus chitosan derivatives alone or DNA plus CA plus Lipofectamine 2000. The graph is normalized to the Lipofectamine 2000 only control.

FIG. 8D depicts another example of the luciferase activity in Caco2 cells transfected by DNA plus chitosan derivatives alone or DNA plus CA plus Lipofectamine 2000. The graph is normalized to the Lipofectamine 2000 only control.

FIG. 8E depicts an example of the luciferase activity in A431 cells transfected by DNA plus chitosan derivatives alone or DNA plus CA plus Lipofectamine 2000. The graph is normalized to the Lipofectamine 2000 only control.

FIG. 9A depicts sensitization of Caco2 cells by chitosan derivatives. The graph is normalized to the Lipofectamine 2000 transfection of HEK293T cells.

FIG. 9B depicts sensitization of A549 cells by chitosan derivatives. The graph is normalized to the Lipofectamine 2000 transfection of HEK293T cells.

FIG. 10 depicts an example of the luciferase activity in adipose derived stem cells (ADSC) transfected by DNA plus chitosan derivative, and DNA plus chitosan derivative plus Lipofectamine 2000.

FIG. 11A depicts an example of transfection in CHO-K1 cells with DNA plus chitosan derivative. Transfection is measured by the amount of IgG in the culture medium.

FIG. 11B depicts an example of transfection of CHO-K1 cells where transfection is measured by the amount of SEAP activity in the culture medium.

### DETAILED DESCRIPTION

### Functionalized Chitosan Derivatives

Methods, compounds and compositions for binding and delivering a nucleic acid (e.g., to a cell) are described herein.

Chitosan is derived from chitin, which is a polymer of N-acetylglucosamine that is the main component of the exoskeletons of crustaceans (e.g. shrimp, crab, lobster). Chitosan is formed from chitin by deacetylation, and as such is not a single polymeric molecule, but a class of molecules having different molecular weights and different degrees of deacetylation. The percent deacetylation in commercial chitosans is typically between 50-100%. The chitosan derivatives described herein are generated by functionalizing the resulting free amino groups with positively charged moieties, as described herein. The derivatized chitosans described herein have a number of properties which are advantageous for a nucleic acid delivery vehicle including: they effectively bind and complex the negatively charged nucleic acids, they can be formed into nanoparticles of a controllable size, they be taken up by the cells and they can release the nucleic acids at the appropriate time within the cell.

Chitosans with any degree of deacetylation greater than 50% are used in the present invention, with functionalization between 2% and 50%. (Percent functionalization is determined relative to the number of free amino moieties on the chitosan polymer.) The degrees of deacetylation and functionalization impart a specific charge density to the functionalized chitosan derivative. The resulting charge density affects solubility, nucleic acid binding and subsequent release, and interaction with mammalian cell membranes. Thus, in accordance with the present invention, these properties must be optimized for optimal efficacy. Exemplary chitosan derivatives are described in Baker et al; Ser. No. 11/657,382 filed on Jan. 24, 2007, which is incorporated herein by reference.

The chitosan derivatives described herein have a range of molecular weights that are soluble at neutral and physiological pH, and include for the purposes of this invention molecular weights ranging from 5-1,000 kDa. Embodiments described herein are feature lower molecular weight of derivatized chitosans (<25 kDa, e.g., from about 5 to about 25) which can have desirable delivery and transfection properties, and are small in size and have favorable solubilities. A low molecular weight derivatized chitosan is generally more soluble than a higher molecular weight, the former thus producing a nucleic acid/chitosan complex that will release the nucleic acid and provide increased transfection of cells. Much literature has been devoted to the optimization of all of these parameters for chitosan based delivery systems.

The functionalized chitosan derivatives described herein include the following:

(A) Chitosan-arginine compounds;

(B) Chitosan-natural amino acid derivative compounds;

(C) Chitosan-unnatural amino acid compounds;

(D) Chitosan-acid amine compounds; and

(E) Chitosan-guanidine compounds.

(F) Neutral chitosan derivative compounds.

(A) Chitosan-Arginine Compounds

In some embodiments, the present invention is directed to chitosan-arginine compounds, where the arginine is bound through a peptide (amide) bond via its carbonyl to the primary amine on the glucosamines of chitosan:

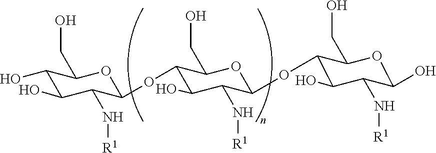

wherein each R¹ is independently selected from hydrogen, acetyl, and a group of the following formula:

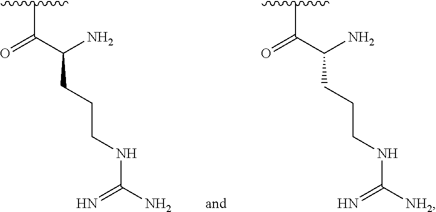

or a racemic mixture thereof,

wherein at least 25% of R¹ substituents are H, at least 1% are acetyl, and at least 2% are a group of the formula shown above.

(B) Chitosan-Natural Amino Acid Derivative Compounds

In some embodiments, the present invention is directed to chitosan-natural amino acid derivative compounds, wherein the natural amino acid may be histidine or lysine. The amino is bound through a peptide (amide) bond via its carbonyl to the primary amine on the glucosamines of chitosan:

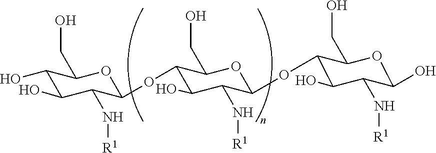

wherein each R¹ is independently selected from hydrogen, acetyl, and a group of the following formula:

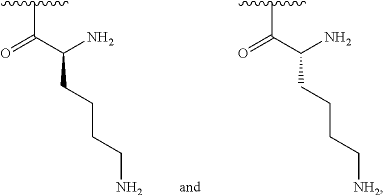

or a racemic mixture thereof, wherein at least 25% of R¹ substituents are H, at least 1% are acetyl, and at least 2% are a group of the formula shown above; OR a group of the following formula:

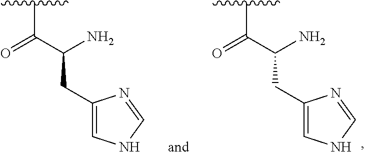

or a racemic mixture thereof, wherein at least 25% of R¹ substituents are H, at least 1% are acetyl, and at least 2% are a group of the formula shown above.

(C) Chitosan-Unnatural Amino Acid Compounds

In some embodiments, the present invention is directed to chitosan-unnatural amino acid compounds, where the unnatural amino acid is bound through a peptide (amide) bond via its carbonyl to the primary amine on the glucosamines of chitosan:

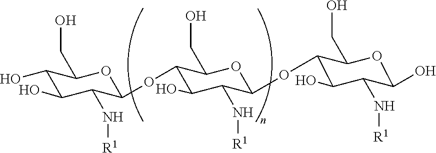

wherein each R³ is independently selected from hydrogen, acetyl, and a group of the following formula:

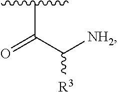

wherein R³ is an unnatural amino acid side chain, and wherein at least 25% of R¹ substituents are H, at least 1% are acetyl, and at least 2% are a group of the formula shown above.

Unnatural amino acids are those with side chains not normally found in biological systems, such as ornithine (2,5-diaminopentanoic acid). Any unnatural amino acid may be used in accordance with the invention. In some embodiments, the unnatural amino acids coupled to chitosan have the following formulae:

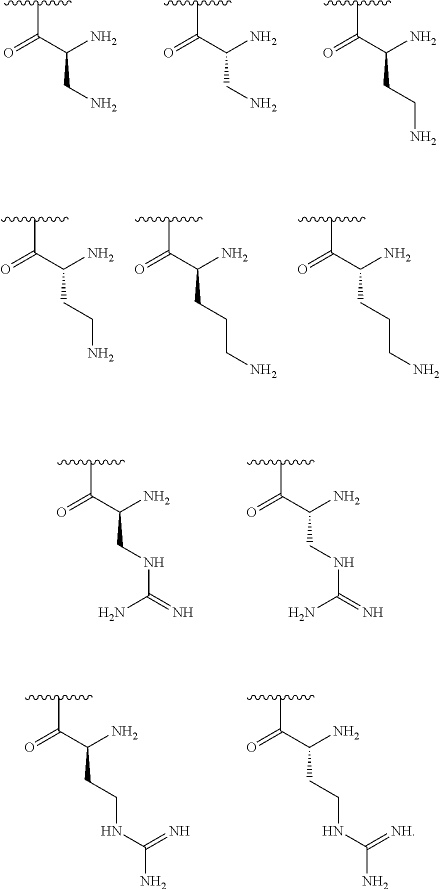

(D) Chitosan-Acid Amine Compounds

In some embodiments, the present invention is directed to chitosan-acid amine compounds, or their guanidylated counterparts. The acid amine is bound through a peptide (amide) bond via its carbonyl to the primary amine on the glucosamines of chitosan:

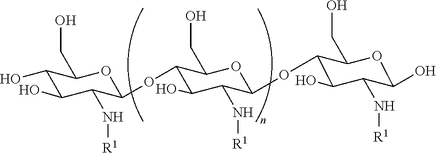

wherein each R¹ is independently selected from hydrogen, acetyl, and a group of the

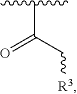

wherein R³ is selected from amino, guanidino, and C₁-C₆ alkyl substituted with an amino or a guanidino group, wherein at least 25% of R¹ substituents are H, at least 1% are acetyl, and at least 2% are a group of the formula shown above

In some embodiments, R¹ is selected from one of the following:

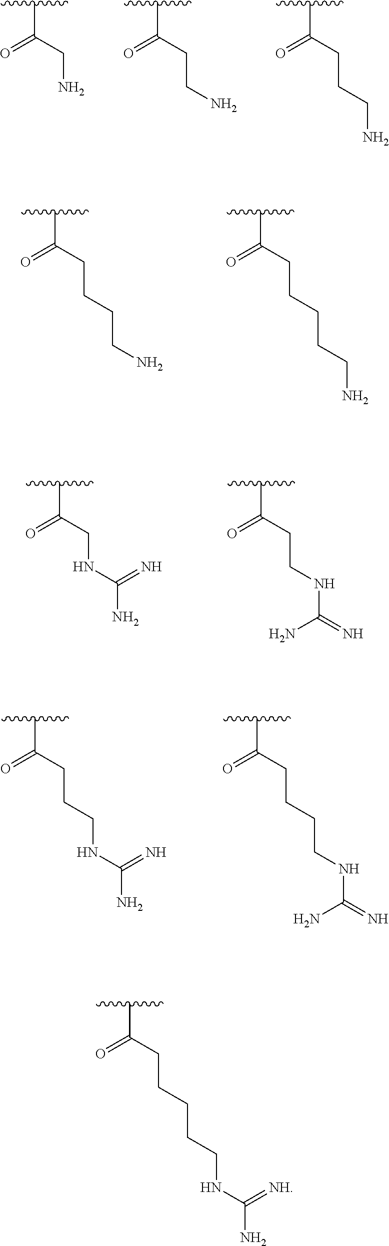

(E) Chitosan-Guanidine Compounds

In some embodiments, the present invention is directed to chitosan-guanidine compound

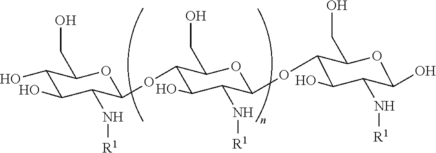

wherein each R¹ is independently selected from hydrogen, acetyl, and a group in which R¹, together with the nitrogen to which it is attached, forms a guanidine moiety; wherein at least 25% of R¹ substituents are H, at least 1% are acetyl, and at least 2% form a guanidine moiety together with the nitrogen to which it is attached.

(F) Neutral Chitosan Derivative Compounds

In some embodiments, the present invention is directed to neutral chitosan derivative compounds. Exemplary neutral chitosan derivative compounds include those where one or more amine nitrogens of the chitosan has been covalently attached to a neutral moiety such as a sugar:

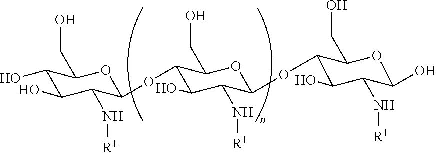

wherein each R¹ is independently selected from hydrogen, acetyl, and a sugar (e.g., a naturally occurring or modified sugar) or an α-hydroxy acid. Sugars can be monosaccharides, disaccharides or polysaccharides such as glucose, mannose, lactose, maltose, cellubiose, sucrose, amylose, glycogen, cellulose, gluconate, or pyruvate. Sugars can be covalently attached via a spacer or via the carboxylic acid, ketone or aldehyde group of the terminal sugar. Examples of α-hydroxy acids include glycolic acid, lactic acid, and citric acid. In some preferred embodiments, the neutral chitosan derivative is chitosan-lactobionic acid compound or chitosan-glycolic acid compound. Exemplary salts and coderivatives include those known in the art, for example, those described in US 2007/0281904, the contents of which is incorporated by reference in its entirety.

### Nucleic Acids

Methods, compounds and compositions for binding and delivering a nucleic acid (e.g., to a cell) are described herein. The nucleic acids (or polynucleotides) described herein include, e.g., deoxyribonucleic acid (DNA) and ribonucleic acid (RNA). DNA may be in form of cDNA, in vitro polymerized DNA, plasmid DNA, parts of a plasmid DNA, genetic material derived from a virus, linear DNA, expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), shRNA (small hairpin RNA), miRNA (microRNA), piRNA (Piwi-interacting RNA), long non-coding RNA, RNA derived from a virus, ribozymes, or derivatives of these groups. Anti-sense is a polynucleotide that interferes with the function of DNA and/or RNA. In addition these forms of DNA and RNA may be single, double, triple, or quadruple stranded. The term also includes PNAs (peptide nucleic acids), phosphorothioates, and other variants of the phosphate backbone of native nucleic acids.

### Applications of Nucleic Acid Delivery

Methods, compounds and compositions for binding and delivering a nucleic acid (e.g., to a cell) are described herein. A nucleic acid can be delivered (e.g., transfected) to a cell to express an exogenous nucleotide sequence, to inhibit, eliminate, augment, or alter expression of an endogenous nucleotide sequence, or to affect a specific physiological characteristic not naturally associated with the cell. The nucleic acid can be a sequence whose presence or expression in a cell alters the expression or function of cellular genes or RNA. A delivered nucleic acid can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A nucleic acid based gene expression inhibitor comprises any nucleic acid containing a sequence whose presence or expression in a cell causes the degradation of or inhibits the function, transcription, or translation of a gene in a sequence-specific manner. Exemplary nucleic acid based expression inhibitors include, e.g., siRNA, microRNA, interfering RNA or RNAi, dsRNA, ribozymes, antisense polynucleotides, and DNA expression cassettes encoding siRNA, microRNA, dsRNA, ribozymes or antisense nucleic acids. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

A nucleic acid can be delivered (e.g., transfected) to a cell to study gene function. Delivery of a nucleic acid to a cell can also have clinical applications. Clinical applications include, e.g., treatment of cancers, neurodegenerative disorders, infectious disorders, muscle disorders or injury, cardiovascular disorders, endocrine disorders, immune modulation and vaccination, and metabolic disorders (see, e.g., Baumgartner et al. 1998, Blau et al. 1995, Svensson et al. 1996, Baumgartner et al. 1998, Vale et al. 2001, Simovic et al. 2001).

### Specific Genes or Targets

As used herein, “therapeutic transgene” refers to a nucleic acid, the expression of which in the target cell produces a therapeutic effect. Exemplary therapeutic transgenes include, e.g., tumor suppressor genes, antigenic genes, cytotoxic genes, cytostatic genes, pro-drug activating genes, apoptotic genes, pharmaceutical genes or anti-angiogenic genes. The nucleic acids of the present invention may be used to produce one or more therapeutic transgenes, either in tandem through the use of IRES elements or through independently regulated promoters.

As used herein, “tumor suppressor gene” refers to a nucleic acid, the expression of which in the target cell is capable of suppressing the neoplastic phenotype and/or inducing apoptosis. Exemplary tumor suppressor genes include, e.g., the APC gene, the BRCA-1 gene, the BRCA-2 gene, the CDKN2A gene, the DCC gene, the DPC4 (SMAD4) gene, the MADR2/JV18 (SMAD2) gene, the MEN1 gene, the MTS1 gene, the NF1 gene, the NF2 gene, the p16 (INK4A) gene, the p53 gene, the PTEN gene, the Rb gene, the VHL gene, the WRN gene, and the WT1 gene.

As used herein, “antigenic gene” refers to a nucleic acid, the expression of which in the target cells results in the production of a cell surface antigenic protein capable of recognition by the immune system. Exemplary antigenic genes include, e.g., carcinoembryonic antigen (CEA), and p53. In order to facilitate immune recognition, the antigenic gene may be fused to the MHC class I antigen. Preferably the antigenic gene is derived from a tumor cell specific antigen, e.g., a tumour rejection antigen, such as the MAGE, BAGE, GAGE and DAGE families of tumor rejection antigens.

As used herein, “cytotoxic gene” refers to a nucleic acid, the expression of which in a cell produces a toxic effect. Exemplary cytotoxic genes include, e.g., nucleic acid sequences encoding pseudomonas exotoxin, ricin toxin, and diphtheria toxin.

As used herein, “cytostatic gene” refers to a nucleic acid, the expression of which in a cell produces an arrest in the cell cycle. Exemplary cytostatic genes include, e.g., the p21 gene, the Rb gene, the E2F gene, the genes encoding cyclin-dependent kinase inhibitors such as P16, p15, p18 and p19, and the growth arrest specific homeobox (GAX) gene.

As used herein, “cytokine gene” refers to a nucleic acid, the expression of which in a cell produces a cytokine. Exemplary cytokines include, e.g., GM-CSF, the interleukins, e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-26, IL-27, IL-28, IL-29, IL-30, IL-31, IL-32, IL-33, IL-35, interferons of the α, β, and γ subtypes.

As used herein, “chemokine gene” refers to a nucleic acid, the expression of which in a cell produces a chemokine. Chemokines are a group of structurally related low-molecular weight factors secreted by cells having mitogenic, chemotactic or inflammatory activities. These proteins can be sorted into two groups based on the spacing of the two amino-terminal cysteines. In the first group, the two cysteines are separated by a single residue (C-x-C), while in the second group, they are adjacent (C—C). Examples of member of the ‘C-x-C’ chemokines include, e.g., platelet factor 4 (PF4), platelet basic protein (PBP), interleukin-8 (IL-8), melanoma growth stimulatory activity protein (MGSA), macrophage inflammatory protein 2 (MIP-2), mouse Mig (m119), chicken 9E3 (or pCEF-4), pig alveolar macrophage chemotactic factors I and II (AMCF-I and -II), pre-B cell growth stimulating factor (PBSF), and TP10. Examples of members of the ‘C—C’ group include, e.g., monocyte chemotactic protein 1 (MCP-1), monocyte chemotactic protein 2 (MCP-2), monocytechemotactic protein 3 (MCP-3), monocyte chemotactic protein 4 (MCP-4), macrophage inflammatory protein 1α (MIP-1-α), macrophage inflammatory protein 1β. (MIP-1-β), macrophage inflammatory protein 1-γ (MIP-1-γ), macrophage inflammatory protein 3α (MIP-3-α, macrophage inflammatory protein 3β(MIP-3-β), chemokine (ELC), macrophage inflammatory protein-4 (MIP-4), macrophage inflammatory protein 5 (MIP-5), LD78β, RANTES, SIS-epsilon (p500), thymus and activation-regulated chemokine (TARC), eotaxin, 1-309, human protein HCC-1/NCC-2, human protein HCC-3, mouse protein C10.

As used herein, “pharmaceutical protein gene” refers to a nucleic acid, the expression of which results in the production of protein have pharmaceutically effect in the target cell. Examples of such pharmaceutical genes include, e.g., the proinsulin gene and analogs, growth hormone gene, dopamine, serotonin, epidermal growth factor, GABA, ACTH, NGF, VEGF, and thrombospondin. Also, the pharmaceutical protein gene may encompass immunoreactive proteins such as antibodies, Fab fragments, Fv fragments, humanized antibodies, chimeric antibodies, single chain antibodies, and human antibodies derived from non-human sources.

As used herein, “pro-apoptotic gene” refers to a nucleic acid, the expression thereof results in the induction of the programmed cell death pathway of the cell. Examples of pro-apoptotic genes include, e.g., p53, adenovirus E3 and E4 genes, p53 pathway genes, and genes encoding the caspases.

As used herein, “pro-drug activating genes” refers to a nucleic acid, the expression of which, results in the production of protein capable of converting a non-therapeutic compound into a therapeutic compound, which renders the cell susceptible to killing by external factors or causes a toxic condition in the cell. Example of a pro-drug activating genes include, e.g., cytosine deaminase gene, and thymidine kinase (TK) gene.

As used herein, “anti-angiogenic” genes refers to a nucleic acid, the expression of which results in the extracellular secretion of anti-angiogenic factors. Exemplary anti-angiogenesis factors include angiostatin, inhibitors of vascular endothelial growth factor (VEGF) such as Tie 2, and endostatin.

### Compositions

Methods, compounds and compositions for binding and delivering a nucleic acid (e.g., to a cell) are described herein. Nucleic acids may be delivered in vivo or in vitro. Accordingly, compositions for nucleic acid delivery are described herein.

In some embodiments, a composition for nucleic acid delivery includes a functionalized chitosan-arginine described herein, e.g., a compound of formula (I). The positively charged moieties on the polymer serve to effectively bind the negatively charged nucleic acids.

In some embodiments, the compositions include a nucleic acid, e.g., a nucleic acid described herein.

In some embodiments, the compositions include a compound that is used to promote transfection. Such compounds may include polysaccharides such as diethylaminoethyl-Dextran (DEAE-Dextran), salts such as calcium phosphate (e.g., HEPES-buffered saline solution (HeBS) containing phosphate ions combined with calcium chloride), lipids (e.g., cationic lipids or phospholipids), formulations of lipids, cationic polymers (e.g., polylysine or polyethyleneimine (PEI)), multicomponent nonliposomal reagents (e.g., lipids, polymers and combinations thereof), or nanoparticles of an inert solid (e.g., gold).

In some embodiments, the compositions include a precipitating solution, which may include salts such as sodium sulfate or a tripolyphosphate (TPP) salt. The pH, ionic strength and temperature of the precipitating solutions can be adjusted for optimization of binding and delivery, the range of DNA incorporation at pH 7 with minimal coprecipitating factors is facilitated and optimized by incorporation of the described positively charged chitosan derivatives. Due to the solubility of the chitosan derivatives at a range of molecular weights and degrees of functionalization, optimization of a delivery strategy for a variety of nucleic acid types and sizes is facilitated.

### Methods of Making Derivatized Chitosan/Nucleic Acid Complexes

The chitosan derivative/nucleic acid complexes described herein can be made (e.g., formed) by various methods. In some embodiments, the complex comprises a particle, wherein the particle comprises a chitosan derivative and a nucleic acid. In some embodiments, the particle is nanometers in dimension, for example, due to the nature of the molecules involved, e.g. the chitosan derivative and/or the nucleic acid. In one embodiment, the chitosan derivative/nucleic acid complex is made (e.g., formed) by mixing a chitosan derivative (e.g., a chitosan derivative described herein (e.g., chitosan-arginine)) with nucleic acid (e.g., DNA) at a chitosan derivative:nucleic acid ratio described herein in H₂O (e.g., H₂O at neutral pH) (e.g., as described in Qi et al., Carbohydrate Research 339 (2004):2693-2700), the content of which is incorporated herein by reference in its entirety). In another embodiment, the chitosan derivative/nucleic acid complex is made (e.g., formed) by premixing nucleic acid (e.g., DNA) with a chitosan derivative (e.g., a chitosan derivative described herein (e.g., chitosan-arginine) at a ratio described herein in a medium (e.g., a serum-free medium). In some embodiments, the nucleic acid is added to the medium before the chitosan derivative is added. In some embodiments, the chitosan derivative is added to the medium before the nucleic acid is added. In yet another embodiment, the chitosan derivative/nucleic acid complex is made (e.g., formed) by sequentially adding nucleic acid (e.g., DNA) and a chitosan derivative (e.g., a chitosan derivative described herein (e.g., chitosan-arginine) at a ratio described herein to the cells. In some embodiments, the nucleic acid is added to the cells before the chitosan derivative is added. In some embodiments, the chitosan derivative is added to the cells after the nucleic acid is added. In some embodiments, the cells are suspension cultured cells. In one embodiment, the method further comprising the step of adding a lipid or lipid formulation (e.g., Lipofectamine 2000) to the chitosan derivative/nucleic acid mixture or adding a lipid or lipid formulation (e.g., Lipofectamine 2000) to the cells.

### Nanoparticle Complexes

Methods and compositions described herein are useful for the formation and use of a nanoparticle complex of controllable size having a composition including the chitosan derivative and nucleic acid. The nanoparticle complexes may include but are not limited to coprecipitate(s) such as sodium sulfate or tripolyphosphate (TPP) salt. The nanoparticle complexes are taken up by a cell where the nucleic acid is therein released in a desirable timeframe.

### Transfection

Methods, compounds and compositions for binding and delivering a nucleic acid (e.g., to a cell) are described herein. In some embodiments, a composition or particle described herein may be delivered in vivo via transfection.

Transfection is the process of introducing nucleic acids into cells, e.g., animal cells, by non-viral methods. Transfection of animal cells typically involves opening transient pores or ‘holes’ in the cell plasma membrane, to allow the uptake of material. Genetic material (such as supercoiled plasmid DNA or siRNA constructs), or even proteins such as antibodies, may be transfected. In addition to electroporation, transfection can be carried out by mixing a cationic lipid with the material to produce liposomes, which fuse with the cell plasma membrane and deposit their cargo inside.

In transient transfection, the DNA introduced in the transfection process is usually not inserted into the nuclear genome, and the foreign DNA is lost at the later stage when the cells undergo mitosis. In stable transfection, the transfected gene actually remains in the genome of the cell and its daughter cells. To accomplish this, another gene is co-transfected, which gives the cell some selection advantage, such as resistance towards a certain toxin. Some (very few) of the transfected cells will, by chance, have inserted the foreign genetic material into their genome. If the toxin, towards which the co-transfected gene offers resistance, is then added to the cell culture, only those few cells with the foreign genes inserted into their genome will be able to proliferate, while other cells will die. After applying this selection pressure for some time, only the cells with a stable transfection remain and can be cultivated further.

### Methods of Transfection

Methods, compounds and compositions for binding and delivering a nucleic acid (e.g., to a cell) are described herein. In some embodiments, a composition or particle described herein may be delivered in vivo via transfection. There are various methods of introducing foreign nucleic acids into a eukaryotic cell. Many materials can be used as carriers for transfection. Exemplary methods of transfection include, for example:

DEAE-Dextran: Diethylaminoethyl-Dextran (DEAE-Dextran), a polycationic derivative of Dextran, associates tightly with the negatively charged nucleic acid, and carries it into the cell.

Calcium phosphate: HEPES-buffered saline solution (HeBS) containing phosphate ions is combined with a calcium chloride solution containing the DNA or RNA to be transfected. When the two are combined, a fine precipitate of the positively charged calcium and the negatively charged phosphate will form, binding the nucleic acid to be transfected on its surface. The suspension of the precipitate is then added to the cells to be transfected (e.g., a cell culture grown in a monolayer). The cells take up at least some of the precipitate, and with it, the nucleic acid.

Liposome: A liposome is a tiny bubble (vesicle), made out of the same material as a cell membrane. Cell membranes are usually made of phospholipids, which are molecules that have a hydrophilic head and a hydrophobic tail. When membrane phospholipids are disrupted, they can reassemble themselves into liposomes as bilayers or monolayers. Liposomes can fuse with the cell membrane, then release the nucleic acid into the cell.

Cationic polymers: Cationic polymers, such as polylysine and polyethyleneimine (PEI) interact with nucleic acid to form small complexes and the complex is taken up by the cell via endocytosis, then released.

Non-liposomal lipid-based: Multicomponent, nonliposomal reagents consisting of lipids, polymers and combinations thereof. Non-liposomal lipids form micelles of uniform size with nucleic acid that interact with the cell membrane. The complex is taken up by the cell via endocytosis, then released.

Nanoparticle: A nanoparticle of an inert solid (e.g., gold) is coupled to the nucleic acid, and then “shot” directly into the target cell's nucleus by a gene gun.

Electroporation: Electroporation or electropermeabilization, is a significant increase in the electrical conductivity and permeability of the cell plasma membrane caused by an externally applied electrical field, therefore introduce nucleic acids into a cell.

Nucleofection: Based on the physical method of electroporation, nucleofection uses a combination of optimized electrical parameters, generated by a special device called Nucleofector, with cell-type specific reagents. The substrate is transferred directly into the cell nucleus and the cytoplasm.

Sonoporation: Sonoporation utilizes the interaction of ultrasound (US) with the cell to temporarily permeabilize the cell membrane allowing for the uptake of nucleic acid from the extracellular environment.

Heat shock: heat shock is the effect of subjecting a cell to a higher temperature than that of the ideal body temperature of the organism from which the cell line was derived. The sudden change in temperature causes the cell membrane pores to open up to larger sizes, allowing nucleic acid to enter. After a brief interval, the cells are quickly cooled to a low temperature again. This closes up the pores, and traps the DNA inside.

Magnetofection: Magnetofection uses magnetic fields to concentrate and transport particles containing nucleic acid into the target cells.

### Exemplary Transfection Reagents/Kits

In vivo transfection reagents/kits include, e.g., MaxSuppressor™ RNA-LANCEr, TransIT® In Vivo Gene Delivery System, TransIT®-EE Delivery Solution, TransIT®-EE Starter Kit, TransIT®-QR Delivery Solution, TransIT®-QR Starter Kit, TransPass™ P Protein Transfection Reagent, in vivo-jetPEI™ Delivery Reagent, jetSI™ siRNA Delivery Reagent, in vivo-jetPEI™-Gal Delivery Reagent, and in vivo-jetPEP™-Man Delivery Reagent.

Liposome transfection reagents/kits include, e.g., SureFECTOR, UniFECTOR, PlasFect™, RiboFect™, Nupherin™ Transfection Reagent, Lipofectamine™ 2000 CD Transfection Reagent, Optifect™ Transfection Reagent, Lipofectamine™ 2000 Reagent, Lipofectamine™ LTX Reagent, Lipofectamine™ Reagent, Lipofectin Reagent, LyoVec™, HiFect™, n-Blast Transfection Reagent, n-Fect™ Neuro Transfection Reagent, n-Fect™ Transfection Reagent, p-Fect™ Transfection Reagent, TransPass™ D1 Transfection Reagent, EcoTransfect, DreamFect™, Tfx™ Reagents Transfection Trio, Tfx™-50 Reagent, Tfx™-10 Reagent, Tfx™-20 Reagent, TransFast™ Tfx™ Transfection Reagent, Transfectam™ Reagent for the Transfection of Eukaryotic Cells, DOSPER™ Liposomal Transfection Reagent, DOTAP™ Liposomal Transfection Reagent, X-tremeGene™ Q2 Transfection Reagent, DOTAP™ methosulfate, ESCORT™ II Transfection Reagent, ESCORT™ III Transfection Reagent, ESCORT™ IV Transfection Reagent, ESCORT™ Transfection Reagent, GenJet™ DNA In Vitro Transfection Reagent, GenJet™ (Ver. II) DNA In Vitro Transfection Reagent, GenJet™ Plus DNA In Vitro Transfection Reagent, LipoD293™ (Ver. II) DNA In Vitro Transfection Reagent, LipoJet™ (Ver. II) DNA In Vitro Transfection Reagent, PolyJet™ DNA In Vitro Transfection Reagent, Targefect™ F-1, Genetransfer™, and HMG-1,2 Mixture.

Magnetic transfection reagents/kits include e.g., NIMT®FeOfection, MA Lipofection Enhancer (IBA GmbH), MATra-A, Matra-S Immobilizer, Magnetofection™-ViroMag 100, Magnetofection™-ViroMag 1000, Magnetofection™-ViroMag 200, ViroMag R/L, Magnetofection™-CombiMag, Magnetofection™-PolyMag, Magnetofection™-SilenceMag.

mRNA transfection reagent/kits include, e.g., TransIT®-mRNA.

Non-liposomal transfection reagents/kits include, e.g., Calcium Phosphate transfection reagents/kits, e.g., Calcium Phosphate Transfection Kit (Invitrogen), Mammalian Cell Transfection Kit (Millipore), ProFection® Mammalian Transfection System—Calcium Phosphate, Calcium Phosphate Transfection Kit (Sigma-Aldrich), Mammalian Transfection Kit—Calcium Phosphate (Stratagene), CellPhect Transfection Kit™, Transfection MBS Mammalian Transfection Kit (Stratagene), and CalFectinIM DNA In Vitro Transfection Reagent; Polyethylenimine (PEI) Transfection Kits/Reagents, e.g., Polyethylenimine-Transferrinfection Kit (Bender MedS ystems), jetPEI™ DNA Transfection Reagent, and Polyethylenimine “Max”, (nominally MW 40,000)—High Potency Linear PEI (Polysciences); Polyethylenimine (PEI) Transfection Kits/Reagents (Conjugated), e.g., jetPEI™-FluoF DNA Transfection Reagent, and jetPEI™-RGD DNA Transfection Reagent; and Others, e.g., DNotion Transfection Reagent, GeneChoice®Transfectol™ Transfection Reagent, LipoGen™, Polybrene Infection/Transfection Reagent (Millipore), Transient Expression Transfection Kit (Millipore), TransIT®-Express Transfection Reagent, TransIT®-LT1 Transfection Reagent, TransIT®-LT2 Reagent, TransPass™ D2 Transfection Reagent, GeneJuice® Transfection Reagent, Fecturin™ DNA Transfection Reagent, ProFection® Mammalian Transfection System-DEAE-Dextran, FuGENE® 6 Transfection Reagent, FuGENE® HD Transfection Reagent, MesenFectagen®, DEAE-Dextran Transfection Kit (Sigma-Aldrich), GeneJammer® Transfection Reagent, Mammalian Transfection Kit (Stratagene), SatisFection™ Transfection Reagent, and Targefect F-2.

Oligo Transfection Reagents/Kits include, e.g., TransIT®-Oligo Transfection Reagent, and Oliogfectamine.

Parasite Transfection Reagents/Pathogen Transfection Reagents/Kits include, e.g., Basic Parasite Nucleofector® Kits.

Primary Cell Transfection Reagents/Kits, include, e.g., Cross Species Transfection Reagents/Kits (Primary Cells), e.g., Basic Nucleofector® Kit for Primary Mammalian Endothelial Cells, Basic Nucleofector® Kit for Primary Mammalian Epithelial Cells, Basic Nucleofector® Kit for Primary Mammalian Fibroblasts, Basic Nucleofector® Kit for Primary Mammalian Neurons, TransIT®-Keratinocyte Transfection Reagent, jetPEIT™-Macrophage DNA Transfection Reagent, AstroFectagen® Astrocyte Transfection Kit, EndoFectagen® Endothelial Cell Transfection Kit, EpiFectagen® Epithelial Cell Transfection Kit, FibroFectagen® Fibroblast Transfection Kit, KeratoFectagen® Keratinocyte Transfection Kit, MelanoFectagen® Melanocyte Transfection Kit, NeuroFectagen® Neuron Transfection Kit, and GenJet™ (Ver. II) DNA In Vitro Transfection Reagent for Primary Keratinocytes; Human Cell Transfection Reagents/Kits (Primary Cells), e.g., Human Chondrocyte Nucleofector® Kit, Human Hepatocyte 96-well Nucleofector® Kit, Human CD34 Cell Nucleofector® Kit, Human Mammary Epithelial Cell (HMEC) 96-well Nucleofector Kit, Human Prostate Epithelial Cell (hPrEC) 96-well Nucleofector Kit, Normal Human Bronchial Epithelial Cell (NHBE) 96-well Nucleofector Kit, Human Macrophage Nucleofector® Kit, Human Monocyte 96-well Nucleofector® Kit, Human Monocyte Nucleofector® Kit, Targefect-RAW, Human T Cell Nucleofector® Kit, SMCFectagen® Smooth Muscle Cell Transfection Kit, GenJet™ (Ver. II) DNA In Vitro Transfection Reagent for Smooth Muscle Cell, Targefect-SMC, HUVEC (Human Umbilical Vein Endothelial Cell) Nucleofector® Kit, HUVEC 96-well Nucleofector® Kit, TransPass™ HUVEC Transfection Reagent, jetPEI™-HUVEC DNA Transfection Reagent, GenJet™ (Ver. IT) DNA In Vitro Transfection Reagent for HUVEC, Targefect-HUVEC, and Human Dermal Fibroblast (NHDF) 96-well Nucleofector Kit; Mouse Cell Transfection Reagents/Kits (Primary Cells), e.g., Mouse DC 96-well Nucleofector® Kits, Mouse NSC (Neural Stem Cell) Nucleofector® Kit, and Mouse T Cell 96-well Nucleofector® Kit; and Rat Cell Transfection Reagents/Kits (Primary Cells), e.g., Rat Cardiomyocyte—Neonatal Nucleofector® Kit, and Rat Oligodendrocyte Nucleofector® Kit.

Reverse Transfection Reagents/Kits include, e.g., SureFECT™ Transfection Reagent.

siRNA Transfection Reagents/Kits include, e.g., NIMT®FeOfection, MATra-si Reagent, Magnetofection™-CombiMag, Magnetofection™-PolyMag, Magnetofection™-SilenceMag, RNotion Transfection Reagent, Silencer™ siRNA Transfection II Kit, siPORT™ NeoFX™ Transfection Agent, siPORT™ XP-1 Transfection Agent, siPORT™ Amine Transfection Agent, siPORT™ Lipid Transfection Agent, siPORT™ NeoFX™ Transfection Agent, siFECTOR, NIMT®FeOfection|PURPLE, Transfection reagent (IMGENEX), BLOCK-iT™ Transfection Kit, Lipofectamine™ RNAiMAX, Oligofectamine™ Reagent, Lipofectamine™ 2000 Reagent, siRNA Test Kit—For Cell Lines and Primary Adherent Cells, siIMPORTER™, TransIT®-siQUEST™ Transfection Reagent, TransIT®-TKO siRNA Transfection Reagent, i-Feet si RNA Transfection Reagent, i-Feet Transfection Kit, TransPass™ R1 Transfection Reagent, TransPass™ R2Transfection Reagent, RiboJuice™ siRNA Transfection Reagent, Lullaby®-siRNA transfection reagent, DreamFect™, INTERFERin™ siRNA Transfection Reagent, jetSI™ siRNA Delivery Reagent, jetSI™-ENDO Transfection Reagent, CodeBreaker™ siRNA Transfection Reagent, X-tremeGENE siRNA Transfection Reagent, siRNA Transfection Reagent (Santa Cruz Biotechnology), N-TER Nanoparticle siRNA Transfection System, GeneEraser™ siRNA transfection reagent, Targefect siRNA kit, DharmaFECT 1 Transfection Reagent, DharmaFECT 1, 2, 3, 4 Transfection Reagents, DharmaFECT 2 Transfection Reagent, DharmaFECT 3 Transfection Reagent, DharmaFECT 4 Transfection Reagent, and DharmaFECT Duo Co-Transfection Reagent.

Stem Cell Transfection Reagents/Kits include, e.g., Human MSC (Mesenchymal Stem Cell) Nucleofector® Kit, and Stemfect™ DNA Plasmid Transfection Polymer.

Cell Line Specific Transfection Reagents/Kits include, e.g., GenJet™ (Ver. II) DNA In Vitro Transfection Reagent for 3LL Cell, TransIT®-3T3 Transfection Kit, GenJet™ (Ver. II) DNA In Vitro Transfection Reagent for NIH3T3 Cell, Human B Cell Nucleofector® Kit, GenJet™ (Ver. II) DNA In Vitro Transfection Reagent for B16-F10 Cells, GenJet™ (Ver. II) DNA In Vitro Transfection Reagent for BHK-21 Cell, GenJet™ (Ver. II) DNA In Vitro Transfection Reagent for C6 Cell, GenJet™ (Ver. II) DNA In Vitro Transfection Reagent for C6 Cell, GenJet™ (Ver. II) DNA In Vitro Transfection Reagent for Ca Ski Cell, GenJet™ (Ver. II) DNA In Vitro Transfection Reagent for Caco-2 Cell, DG44 Transfection Kit, TransIT®-CHO Transfection Kit, GenJet™ (Ver. II) DNA In Vitro Transfection Reagent for CHO Cell, TransIT®-COS Transfection Kit, TransPass™ COS Transfection Reagent, GenJet™ (Ver. II) DNA In Vitro Transfection Reagent for COS Cell, Targefect-COS, GenJet™ (Ver. II) DNA In Vitro Transfection Reagent for CV-1 Cell, GenJet™ (Ver. II) DNA In Vitro Transfection Reagent for D 407 Cell, 293fectin™ Transfection Reagent, TransIT®-293 Transfection Reagent, ViraPack™ Transfection Kit, Targefect-293, TransIT®-HeLaMONSTERT™ Transfection Kit, TransPass™ HeLa Transfection Reagent, GenJet™ (Ver. II) DNA In Vitro Transfection Reagent for Hela Cell, Targefect-Hela, Mouse Hepatocyte Nucleofector® Kit, Rat Hepatocyte Nucleofector® Kit, jetPEIT™-Hepatocyte DNA Transfection Reagent, Targefect-Hepatocyte, GenJet™ (Ver. II) DNA In Vitro Transfection Reagent for HepG2 Cell, GenJet™ (Ver. II) DNA In Vitro Transfection Reagent for Huh-7 Cell, Bac-N-Blue™ Transfection Kit, TransIT®-Insecta Transfection Reagent, Insect GeneJuice® Transfection Reagent, FlyFectin™, FectoFly™ I DNA Transfection Kit, FectoFly™ II DNA Transfection Kit, insFect™ DNA In Vitro Transfection Reagent, TransIT®-Jurkat Transfection Reagent, GenJet™ (Ver. II) DNA In Vitro Transfection Reagent for K-562 Cell, GenJet™ (Ver. II) DNA In Vitro Transfection Reagent for K-562 Cell, GenJet™ (Ver. II) DNA In Vitro Transfection Reagent for L929 Cell, GenJet™ (Ver. II) DNA In Vitro Transfection Reagent for LNCaP Cell, GenJet™ (Ver. II) DNA In Vitro Transfection Reagent for MCF-7 Cell, GenJet™ (Ver. II) DNA In Vitro Transfection Reagent for MDA-MB231Cell, GenJet™ (Ver. II) DNA In Vitro Transfection Reagent for MDCK Cell, MEF Starter Nucleofector® Kit, Targefect-MEF, GenJet™ (Ver. II) DNA In Vitro Transfection Reagent for M-PAC Cell, GenJet™ (Ver. II) DNA In Vitro Transfection Reagent for MRC-5 Cell, GenCarrier-1™ DNA transfection reagent, Cell Line 96-well Nucleofector® Kit SE, Cell Line 96-well Nucleofector® Kit SF, Cell Line 96-well Nucleofector® Kit SG, Cell Line Nucleofector® Kit C, Cell Line Nucleofector® Kit L, Cell Line Nucleofector® Kit R, Cell Line Nucleofector® Kit T, Cell Line Nucleofector® Kit V, Basic Neuron 96-well Nucleofector® Kit, Rat Neuron 96-well Nucleofector® Kit, TransIT®-Neural Transfection Reagent, pn-Fect Transfection Reagent, NeuroPorter™ Transfection Kit, GenJet™ (Ver. II) DNA In Vitro Transfection Reagent for Neuro-2a Cell, GenJet™ (Ver. II) DNA In Vitro Transfection Reagent for NMuMG Cell, GenJet™ (Ver. II) DNA In Vitro Transfection Reagent for NMuMG Cell, GenJet™ (Ver. II) DNA In Vitro Transfection Reagent for PC-3 Cell, TransIT®-Prostate Transfection Kit, GenJet™ (Ver. II) DNA In Vitro Transfection Reagent for SaoS-2 Cell, GenJet™ (Ver. II) DNA In Vitro Transfection Reagent for SHEP Cells, GenJet™ (Ver. II) DNA In Vitro Transfection Reagent for SiHa Cell, GenJet™ (Ver. II) DNA In Vitro Transfection Reagent for SK− OV-3 Cell, GenJet™ (Ver. II) DNA In Vitro Transfection Reagent for U-20S Cell, and VeroFect, GenJet™ (Ver. II) DNA In Vitro Transfection Reagent for WEHI-231 Cell.

### Kits

A compound or composition described herein can be provided in a kit. The kit includes (a) a composition that includes a compound described herein, and, optionally (b) informational material. The informational material can be descriptive, instructional, marketing or other material that relates to the methods described herein and/or the use of the compound described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to use of the compound described herein to treat a disorder described herein.

In one embodiment, the informational material can include instructions to administer the compound described herein in a suitable manner to perform the methods described herein, e.g., in a suitable dose, dosage form, or mode of administration (e.g., a dose, dosage form, or mode of administration described herein). Preferred doses, dosage forms, or modes of administration are parenteral, e.g., intravenous, intramuscular, subcutaneous, intraparenteral, bucosal, sublingual, intraoccular, and topical. In another embodiment, the informational material can include instructions to administer the compound described herein to a suitable subject, e.g., a human, e.g., a human having or at risk for a disorder described herein. For example, the material can include instructions to administer the compound described herein to such a subject.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as computer readable material, video recording, or audio recording. In another embodiment, the informational material of the kit is contact information, e.g., a physical address, email address, website, or telephone number, where a user of the kit can obtain substantive information about an compound described herein and/or its use in the methods described herein. Of course, the informational material can also be provided in any combination of formats.

In addition to a compound described herein, the composition of the kit can include other ingredients, such as a solvent or buffer, a stabilizer, a preservative, and/or a second compound for treating a condition or disorder described herein. Alternatively, the other ingredients can be included in the kit, but in different compositions or containers than the compound described herein. In such embodiments, the kit can include instructions for admixing the compound described herein and the other ingredients, or for using a compound described herein together with the other ingredients.

The compound described herein can be provided in any form, e.g., liquid, dried or lyophilized form. It is preferred that the compound described herein be substantially pure and/or sterile. When the compound described herein is provided in a liquid solution, the liquid solution preferably is an aqueous solution, with a sterile aqueous solution being preferred. When the compound described herein is provided as a dried form, reconstitution generally is by the addition of a suitable solvent. The solvent, e.g., sterile water or buffer, can optionally be provided in the kit.

The kit can include one or more containers for the composition containing the compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is an implantable delivery device.

### Cell Lines

Exemplary cell lines and their applications in transfection include, e.g.,

Patient-derived cells to be reintroduced into the patient for gene therapy. Examples include

1. Autologous stem cells derived from the bone marrow (marrow derived stem cells or mesenchymal stem cells (MSC)) and fat (adipose derived stem cells (ADSC)) (Bajada, S., Mazakova, I., Richardson, J. B., Ashammakhi, N. J. Updates on stem cells and their applications in regenerative medicine. Tissue Eng Regen Med 2:169-183, 2008).
2. Induced pluripotent stem cells (iPSC) generated from somatic cells (Takashi, K. and Yamanaka, S. Induction of pluripotent stem cells from mouse embryonic and adult fibroblast cultures by defined factors. Cell 126: 663-675, 2006).
3. Circulating immune cells isolated from a patients blood, e.g., natural killer T-cells (Imai C, Iwamoto S, Campana D. Genetic modification of primary natural killer cells overcomes inhibitory signals and induces specific killing of leukemic cells. Blood 106:376-83, 2005).
4. Patient derived neural progenitor cells (Storch, A and Schwarz, J. Neural stem cells and neurodegeneration. Curr Opin Investig Drugs 3::774-81, 2002).
   Examples of therapies are as follows. Repairing the genetic defect in diseases such as cystic fibrosis (Mueller, C. and Flotte, T. R. Gene therapy for cystic fibrosis. Clinic Rev Allerg Immunol 35:164-178, 2008) and hemophilia (Youjin, S, and Jun, Y. The treatment of hemophilia A: from protein replacement to AAV-mediated gene therapy. Biotechnol Lett 31:321-328, 2009). A biogengineering approach to introduce exogenous genes, such as growth factors, and placement of the modified stem cells into an area of missing or damaged tissue to increase the robustness of endogenous repair/regeneration responses such as replacement of cartilage with stem cells expressing bone morphogenetic proteins (BMP) (Gafni, Y., Turgeman, G., Liebergal, M., Pelled, G., Gazit, Z., and Gazit, D. Stem cells as vehicles for orthopedic gene therapy. Gene Therapy 11:417-426, 2004). A therapeutic approach using stem cells transfected with exogenous genes to treat chronic inflammatory diseases such as rheumatoid arthritis (van de Loo, F. A. J., and van den Berg, W. B. Gene therapy for rheumatoid arthritis. Rheum Dis Clin North Am 28:127-149, 2002).

Cells for manufacturing recombinant protein therapeutics such as monoclonal antibodies and vaccines including:
1. Chinese hamster ovary cells (CHO) (Hacker, D. L., De Jesus, M., and Wurm, F. M. 25 years of recombinant proteins from reactor-grown cells—Where do we go from here? Biotechnol Adv 27:1023-7, 2009).
2. Insect cell lines (Cox, M. M. J., and Hollister, J. R. FluBlok, A next generation influenza vaccine manufactured in insect cells. Biologicals 37:182-189, 2009).
3. Madin Darby canine kidney cells (MDCK) (Doroshenko, A., and Halperin, S. A. Trivalent MDCK cell culture-derived influenza vaccine Optaflu. Expert Rev Vaccines 8:679-88, 2009).
4. Vero cells (Barrett, P. N., Mundt, W., Kistner, O., and Howard M. K. Vero cell platform in vaccine production: moving towards cell culture-based viral vaccines. Expert Rev Vaccines 8:607-18, 2009).
5. Plant cells (Ko, K., Brodzik, R., and Steplewski, Z. Production of antibodies in plants: approaches and perspectives. Curr Top Microbiol Immunol 332:55-78, 2009).
6. Any other mammalian cell line used in production of recombinant proteins such as Baby Hamster Kidney (BHK21), Human Embryonic Kidney 2933 (HEK 29), human fibrosarcoma (HT1080) and human lymphoma (Namalwa). Durocher, Y., and Butler, M. Expression systems for therapeutic glycoprotein production. Curr Opp Biotechnol 20:700-707, 2009.

Cells for Research Applications

1. Human Embryonic Kidney (HEK293), Baby Hamster Kidney (BHK21) and COS cells are commonly used to generate research level recombinant proteins following transient transfections (Wurm, F., and Bernard, A. Large-scale transient expression in mammalian cells for recombinant protein production. Curr Opp Biotechnol 10:156-159, 1999).
2. Any cell line used in the laboratory for research purposes e.g., 3T3 fibroblasts, Hs68 human foreskin fibroblasts, HGF-1 fibroblasts, A431 epidermal cells, MDCK epithelial cells, tumor derived cell lines, e.g., MDA-MB-231, MCF-7, THP-1 monocytes.
3. Primary cells derived from mammalian sources e.g., neuronal cells, epithelial cells, fibroblast cells, endothelial cells, myocytes, chondrocytes, osteoblasts, leukocytes.
4. Any cell line that is hard to transfect e.g., Caco2, A549, NIH3T3.

### EXAMPLES

As provided in the Examples below, CA and C/A refer to chitosan-arginine. A fraction of the amines of the glucosamine on chitosan are reacted with a single arginine, as apposed to a dimer, trimer or larger polyarginine. This monoargylation of each reacted amine is accomplished by using a protecting group on the primary amine of the arginine upon coupling as described in U.S. patent application Ser. No. 11/657,382, the contents of which are incorporated herein by reference.

### Example 1

### Chitosan-Arginine Binds DNA in Solution

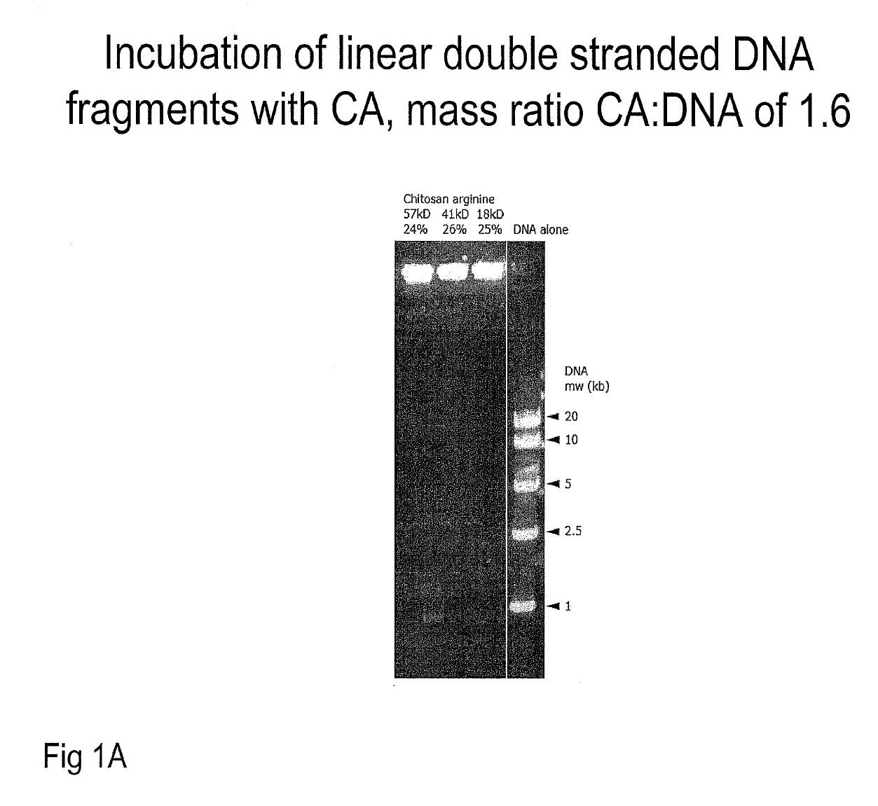

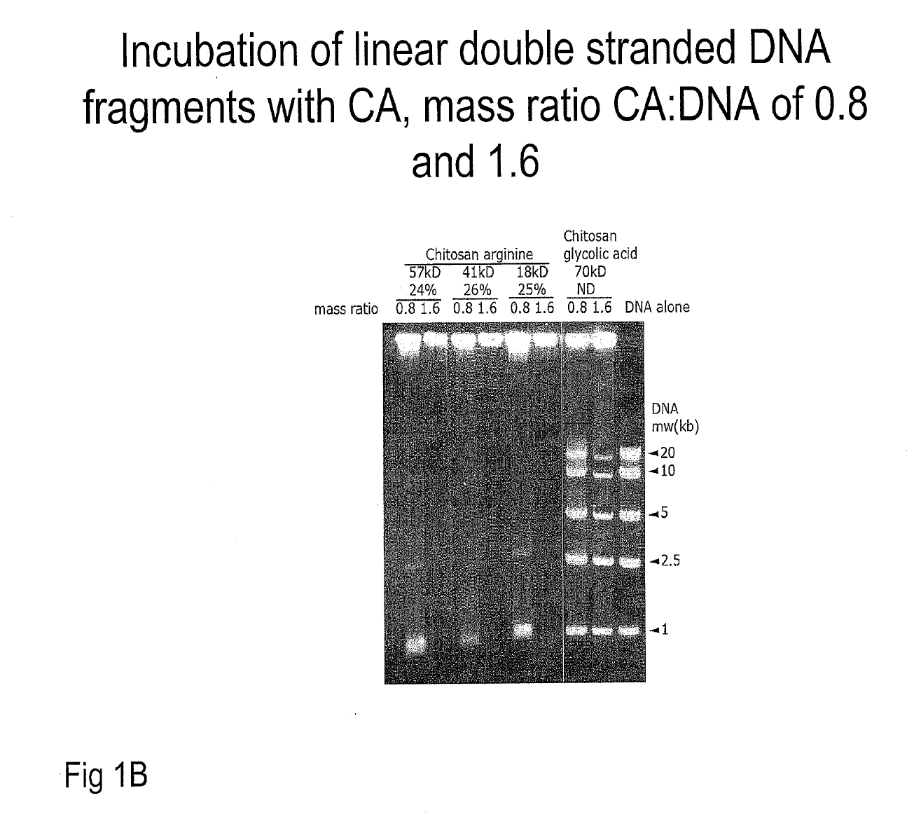

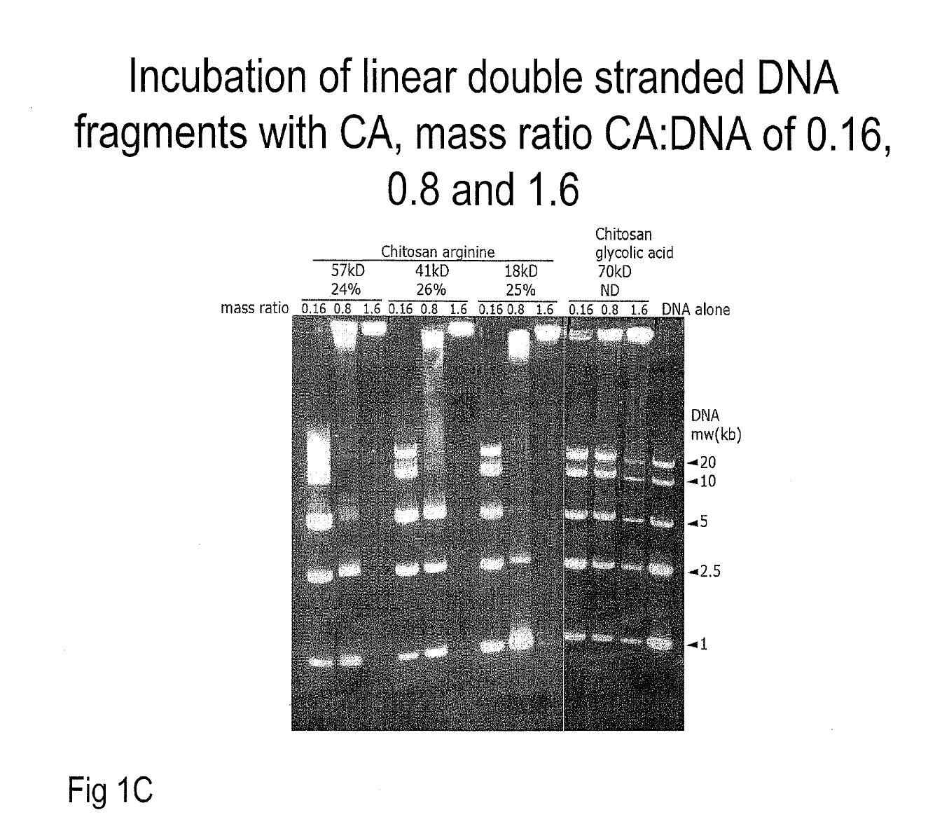

1.25 μg linear fragments of DNA ranging from 1-20 kb were incubated with 2 μg chitosan derivative (chitosan-arginine: 57 kD, 24% functionalization; 41 kD, 26% functionalization; or 18 kD, 25% functionalization; chitosan glycolic acid 70 kD, functionalization not determined), resulting in a chitosan derivative/DNA mass ratio of 1.6, in FIGS. 1A, 1B and 1C), 1 μg chitosan derivative (chitosan derivative/DNA mass ratio of 0.8, in FIGS. 1B and 1C), or 0.2 μg (chitosan derivative/DNA mass ratio of 0.16, in FIG. 1C), in a total volume of 20 μl at room temperature for 30 minutes without agitation. The molecular weight and percent functionalization of each chitosan derivative is shown in FIGS. 1A, 1B and 1C. Loading dye was added to 1×, and electrophoresis was performed on 0.7% agarose in TAE buffer. The percentage of DNA retained in the well is shown in FIGS. 1A, 1B and 1C. These results showed that a significant amount of DNA binds chitosan-arginine in solution.

### Example 2

### Chitosan-Arginine Complexes Transfect HeLa Cells

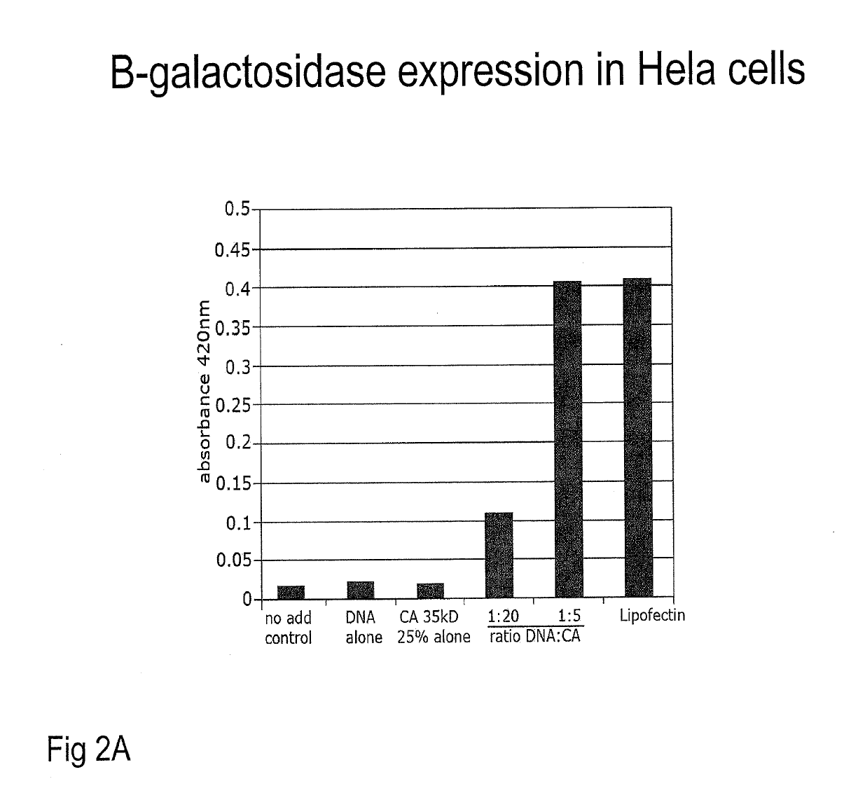

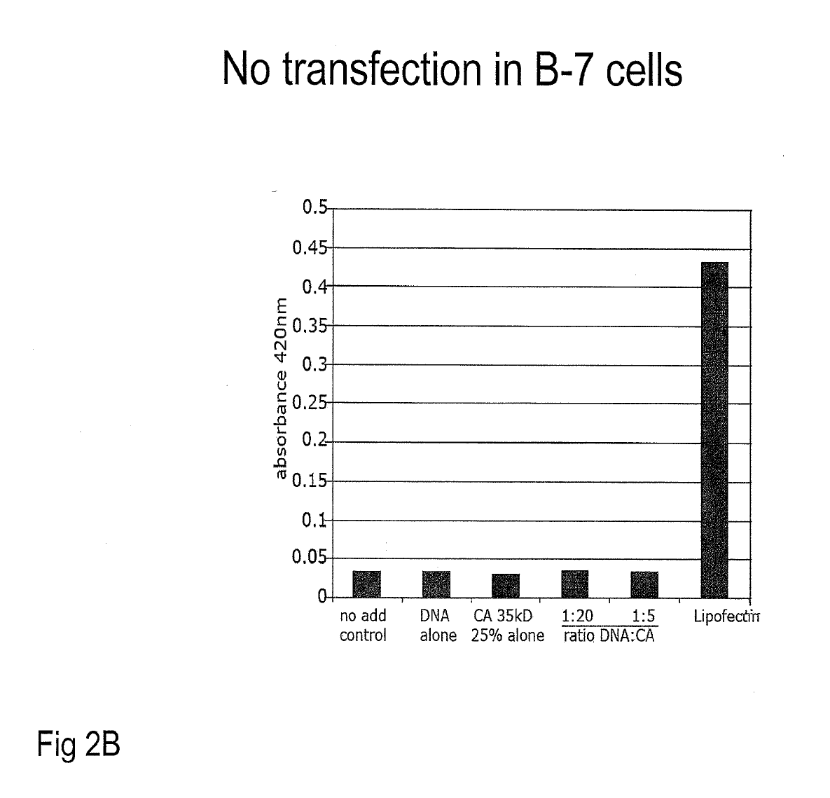

Chitosan-arginine (35 kD, 25% functionalized) was mixed with a plasmid encoding β-galactosidase (pSV-β-galactosidase, Promega) at a DNA:CA ratio of 1:20 and 1:5 in a total volume of 100 μl water at neutral pH to produce complexes according to the methods of Qi et al (Qi, L., Xu, Z., Jiang, X., Hu, C., and Zou, X. Carbohydrate research 339 (2004) 2693-2700). 5 μl of the DNA:chitosan-arginine mix was added to 500 μl of DMEM medium without serum or antibiotics and added to subconfluent Hela cells (FIG. 2A) or B-7 cells (FIG. 2B). Cells were incubated at 37° C. with the transfection mixtures for 24 hours before it was removed and replaced with fresh DMEM medium without serum. Transfection was assessed after three days by measuring the activity of β-galactosidase in cell extracts using o-nitrophenyl-beta-D-galactopyranoside (ONPG) substrate and assaying the absorbance at 420 mM. For the DNA:CA ratio of 1:20 each well of cells received 375 ng DNA and 7.5 μg Chitosan-arginine (35 kD, 25% functionalization), while the 1:5 ratio received 600 ng DNA and 3 μg Chitosan-arginine (35 kD, 25% functionalization). Lipofectin (Invitrogen) transfections were carried out according to the manufacturer's directions. This method resulted in transfection of HeLa cells to approximately the same level as Lipofectin (FIG. 2A) but did not achieve transfection in B-7 cells (FIG. 2B).

### Example 3

### Luciferase Transfection into HEK293T cells and NIH3T3 Cells

4×10⁴ HEK293T or 3×10⁴ NIH3T3 cells in 100 μl of DMEM (all amounts and volumes are given on a per well basis) were seeded into 96 cell plates one day before transfection. Immediately before transfection the medium was replaced with 100 μl/well DMEM supplemented with 10% fetal bovine serum without antibiotics. 0.2 μg/well of a luciferase plasmid with CMV promoter (pGL4.51, Promega) were diluted into DMEM medium (pH7.4). Chitosan derivatives (chitosan-arginine: 57 kD, 24% functionalization; 41 kD, 26% functionalization; or 18 kD, 25% functionalization; chitosan glycolic acid 70 kD, functionalization not determined) were added at a concentration of 100 μg/ml resulting in a DNA:chitosan derivative mass ratio of 1:25. Lipofectamine 2000 (Invitrogen Cat#116680027) was used as the positive control according to the manufacturers directions, at a DNA:Lipofectamine 2000 mass:volume ratio of 1:2.5. All total volumes of mixtures were adjusted to be the same, incubated at room temperature for 20 minutes, then added gently to triplicate wells of cells in 96 well plates. Cells were incubated at 37° C. in a CO₂ incubator for 24 hours prior to testing for luciferase reporter gene expression. After incubation, medium was replaced with PBS containing 5 mM MgCl₂ and 5 mM CaCl₂ and luciferase activity was assayed using a stabilized luciferin substrate (SteadyLite, PerkinElmer). Emitted light was measured using a luminometer (Envision plate reader, PerkinElmer) and expressed as relative light units.

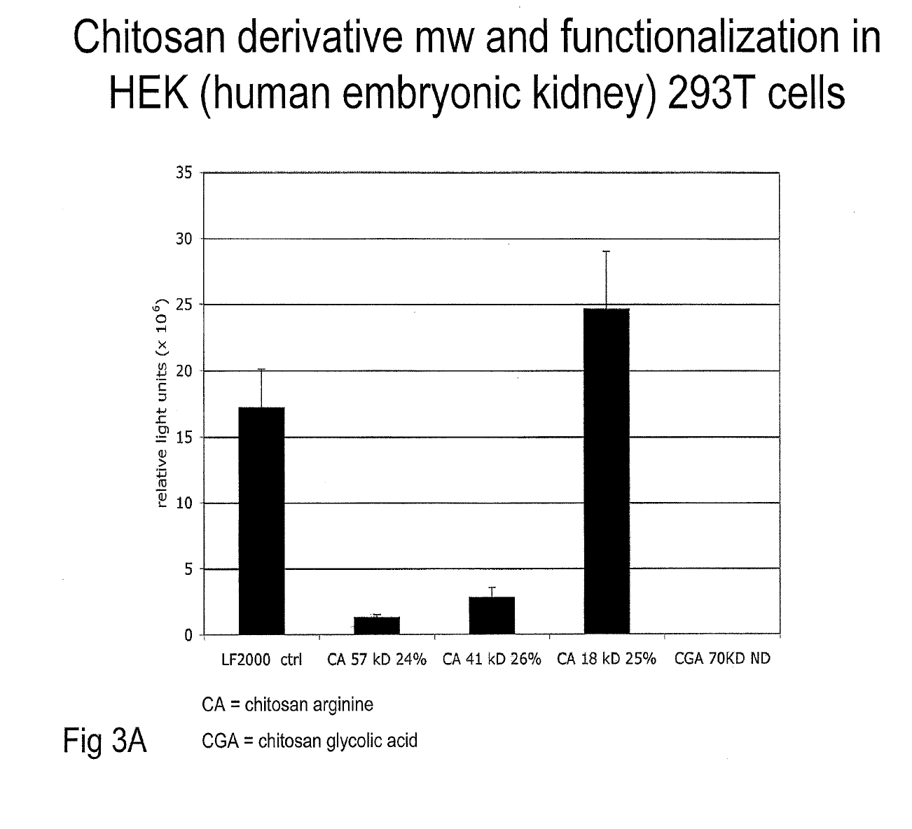

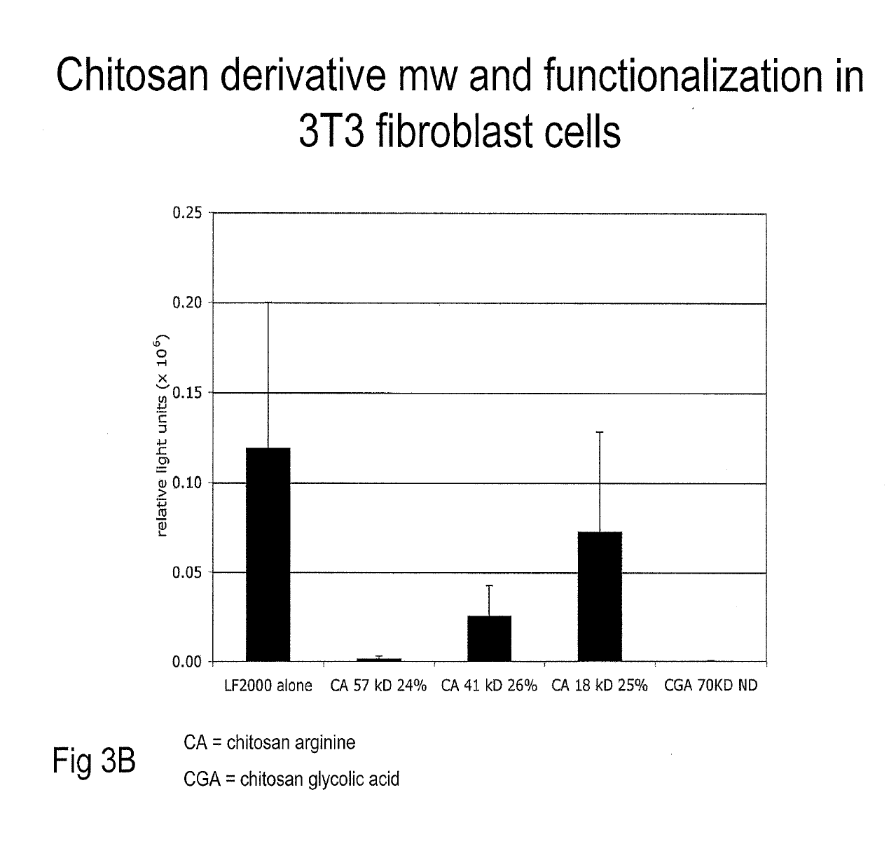

Luciferase activity in HEK293 cells transfected by chitosan derivative is shown in FIG. 3A. Luciferase activity was detected in HEK293T cells transfected by chitosan-arginine: 57 kD, 24% functionalization; 41 kD, 26% functionalization; and 18 kD, 25% functionalization. The transfection efficiency of chitosan-arginine (18 kD, 25% functionalization) was about 1.5 fold higher than that of Lipofectamine 2000. Luciferase activity in NIH3T3 cells transfected with a combination of transfection reagent and chitosan derivative is shown in FIG. 3B. Luciferase activity was detected in NIH3T3 cells transfected by chitosan derivatives (chitosan-arginine: 57 kD, 24% functionalization; 41 kD, 26% functionalization; and 18 kD, 25% functionalization). The transfection efficiency of chitosan-arginine (18 kD, 25% functionalization) was about the same as that of Lipofectamine 2000.

### Example 4

### Optimization of Ratio of Chitosan-Arginine/DNA for Transfection into NIH3T3 Fibroblasts

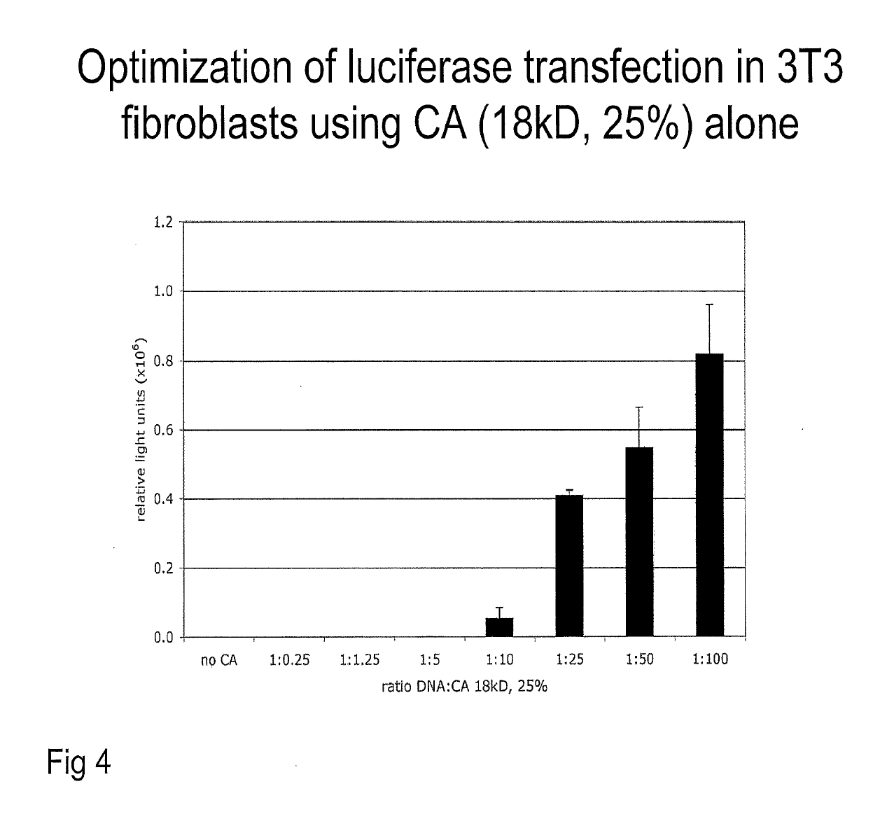

2×10⁴ NIH3T3 cells in 100 μl of DMEM (all amounts and volumes are given on a per well basis) were seeded into 96 cell plates one day before transfection. Immediately before transfection the medium was replaced with 100 μl/well DMEM supplemented with 10% fetal bovine serum without antibiotics. 0.2 μg/well of a luciferase plasmid with CMV promoter (pGL4.51, Promega) were diluted into DMEM medium (pH7.4). Chitosan-arginine (18 kD, 25% functionalization) was added to give DNA mass/Chitosan derivative mass ratio of 1 to 0.25, 1 to 1.25, 1 to 5, 1 to 25, 1 to 50, or 1 to 100. Lipofectamine 2000 was used as the positive control according to the manufacturers directions, at a DNA:Lipofectamine 2000 mass:volume ratio of 1:3. All total volumes of mixtures were adjusted to be the same before transfection. Mixtures were incubated at room temperature for 20 minutes, then added gently to triplicate wells of cells in 96 well plates. Cells were incubated at 37° C. in a CO₂ incubator for 24 hours prior to testing for luciferase reporter gene expression. After incubation, medium was replaced with PBS containing 5 mM MgCl₂ and 5 mM CaCl₂ and luciferase activity was assayed using a stabilized luciferin substrate (SteadyLite, PerkinElmer). Emitted light was measured using a liminometer (Envision plate reader, PerkinElmer) and expressed as relative light units. FIG. 4 shows luciferase expression increases with the amount of chitosan-arginine (18 kD, 25% functionalization) up to a mass ratio of DNA:chitosan-arginine of 1:100.

### Example 5

### Chitosan-Arginine acts as a Transfection Agent when added to Cells Independently of DNA, i.e. no Preincubation Period is Required

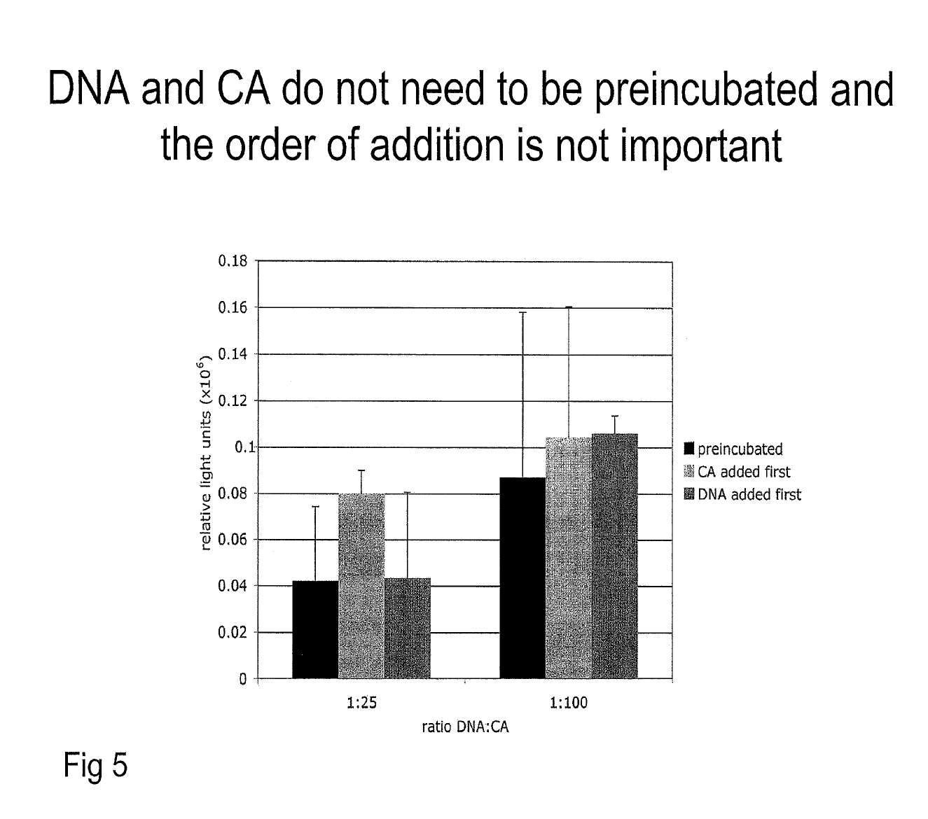

3×10⁴ NIH3T3 cells in 100 μl of DMEM (all amounts and volumes are given on a per well basis) were seeded into 96 cell plates one day before transfection. Immediately before transfection the medium was replaced with 100 μl/well DMEM supplemented with 10% fetal bovine serum without antibiotics. 0.2 μg/well of a luciferase plasmid with CMV promoter (pGL4.51, Promega) were diluted into DMEM medium (pH7.4). Chitosan derivative (18 kD, 25% functionalization) was added to diluted DNA in DMEM medium at a concentration of 100 μg/ml resulting in a DNA:chitosan derivative mass ratio of 1:25, and to a concentration of 200 μg/ml resulting in a DNA:chitosan derivative mass ratio of 1:50. DNA plus chitosan derivative mixtures were incubated at room temperature for 20 minutes, then added gently to triplicate wells of cells. Alternatively, DNA and chitosan derivative were added independently to the cells without any preincubation. All final concentrations of DNA and chitosan derivative and ratios of DNA:chitosan derivative in the cell cultures were the same as those in the preincubated conditions. Cells were incubated at 37° C. in a CO₂ incubator for 24 hours prior to testing for transgene expression. After incubation, medium was replaced with PBS containing 5 mM MgCl₂ and 5 mM CaCl₂ and luciferase activity was assayed using a stabilized luciferin substrate (SteadyLite, PerkinElmer). Emitted light was measured using a luminometer (Envision plate reader, PerkinElmer) and expressed as relative light units. FIG. 5. Equal levels of luciferase activity are achieved by preincubation of DNA and CA, by addition of the CA first followed by DNA within 30 seconds, and by addition of DNA first followed by CA within 30 seconds.

### Example 6

### The Synergistic Effect between Chitosan Derivatives and Lipofectamine 2000 on Transfection of HEK293T and NIH3T3 Cells

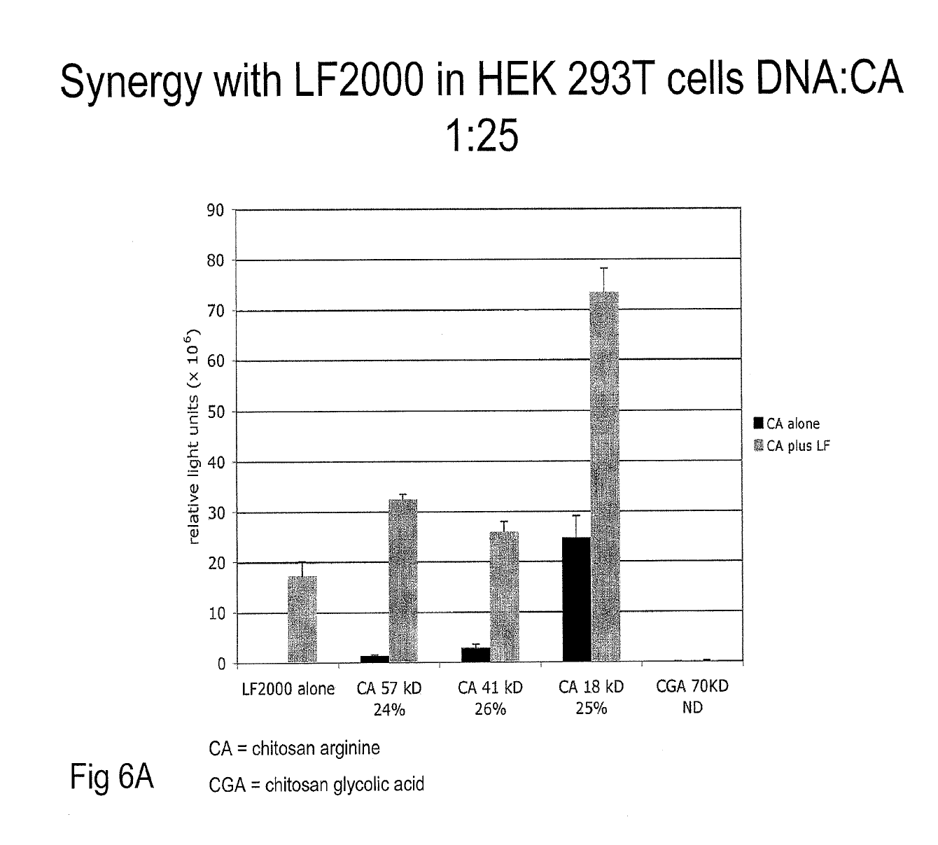

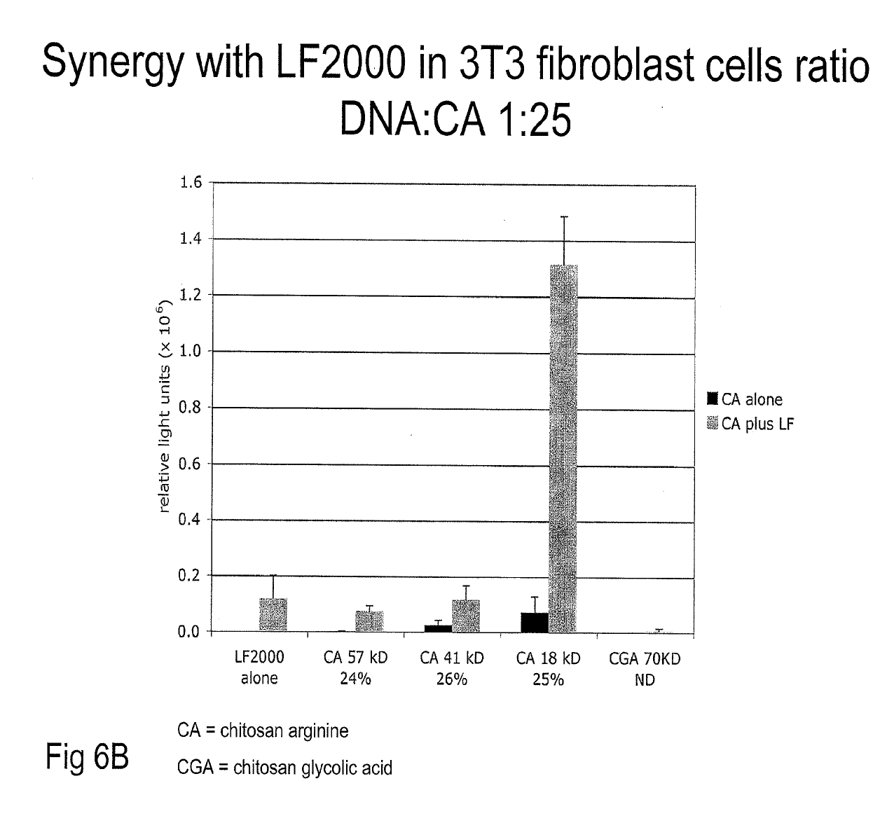

HEK293T and NIH3T3 cells were transfected as described in Examples 3 and 4 using chitosan derivatives (chitosan-arginine: 57 kD, 24% functionalization; 41 kD, 26% functionalization; or 18 kD, 25% functionalization; chitosan glycolic acid 70 kD, functionalization not determined) added at a concentration of 100 μg/ml resulting in a DNA:chitosan derivative mass ratio of 1:25. Additional conditions were prepared that included Lipofectamine 2000 in the preincubation with DNA and CA. Lipofectamine 2000 was used at DNA:Lipofectamine 2000 mass:volume ratio of 1:2.5. The synergistic effect of mixing DNA, chitosan-arginine (18 kD, 25% functionalization) and Lipofectamine 2000 is shown in FIGS. 6A and 6B. In FIG. 6A luciferase activity was detected in HEK293T cells transfected by a combination of DNA and chitosan derivatives (chitosan-arginine: 57 kD, 24% functionalization; 41 kD, 26% functionalization; or 18 kD, 25% functionalization; chitosan glycolic acid 70 kD, functionalization not determined) alone, and with DNA plus chitosan derivatives plus Lipofectamine 2000. The transfection efficiency of a combination of chitosan derivative (chitosan-arginine: 18 kD, 25% functionalization) and Lipofectamine 2000 was about 5 fold higher than that of Lipofectamine 2000 alone and about 3 fold higher than that of chitosan-arginine (18 kD, 25% functionalization) alone. In FIG. 6B Luciferase activity was detected in NIH3T3 cells transfected by a combination of DNA and chitosan derivatives (chitosan-arginine: 57 kD, 24% functionalization; 41 kD, 26% functionalization; and 18 kD, 25% functionalization) alone, and with DNA plus chitosan derivatives plus Lipofectamine 2000. The transfection efficiency of a combination of chitosan derivative (chitosan-arginine:18 kD, 25% functionalization) and Lipofectamine 2000 was about 11 fold higher than that of Lipofectamine 2000 alone and about 19 fold higher than that of chitosan-arginine (18 kD, 25% functionalization) alone.

### Example 7

### Optimization of Ratio of DNA/Chitosan-Arginine for Transfection into NIH3T3 Fibroblasts

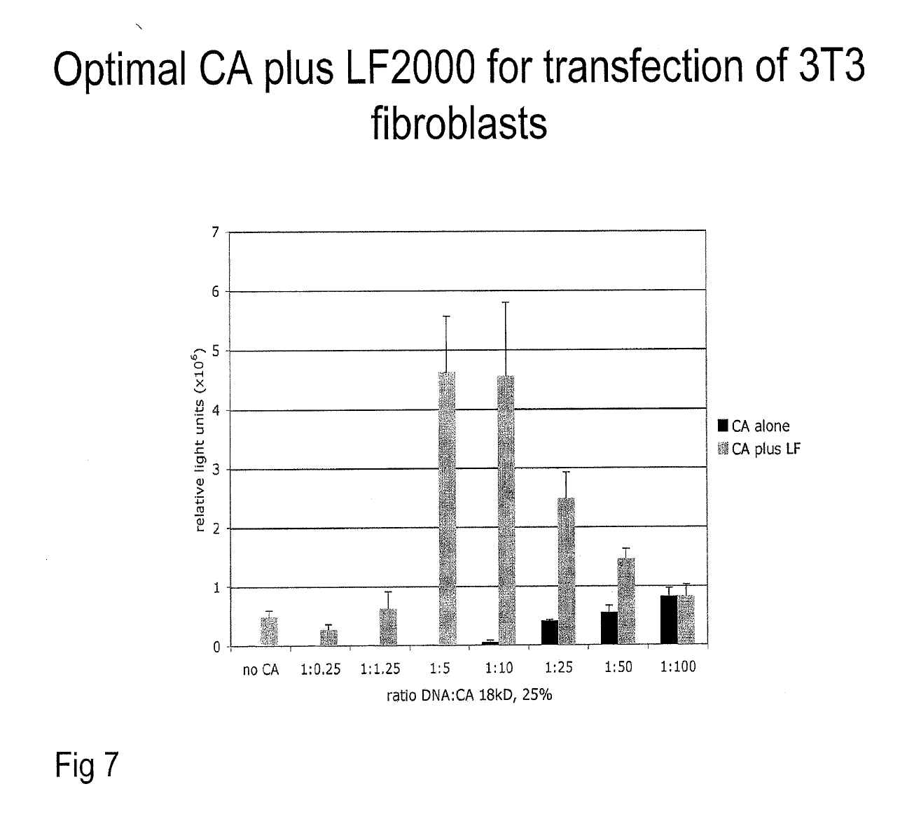

2×10⁴ NIH3T3 cells in 100 μl of DMEM (all amounts and volumes are given on a per well basis) were seeded into 96 cell plates one day before transfection. Immediately before transfection the medium was replaced with 100 μl/well DMEM supplemented with 10% fetal bovine serum without antibiotics. 0.2 μg/well of a luciferase plasmid with CMV promoter (pGL4.51, Promega) were diluted into DMEM medium (pH7.4). Chitosan-arginine (18 kD 25% functionalization) was added to give DNA mass/Chitosan derivative mass ratio of 1 to 0.25, 1 to 1.25, 1 to 5, 1 to 25, 1 to 50, or 1 to 100. Lipofectamine 2000 was used as the positive control, and was added to relevant tubes to test luciferase activity with DNA plus both Lipofectamine 2000 lipid based transfection reagent and chitosan derivative. Lipofectamine 2000 was used at DNA:Lipofectamine 2000 mass:volume ratio of 1:3 All total volumes of mixtures were adjusted to be the same before transfection. Mixtures were incubated at room temperature for 20 minutes, then added gently to triplicate wells of cells in 96 well plates. Cells were incubated at 37° C. in a CO₂ incubator for 24 hours prior to testing for transgene expression. After incubation, medium was replaced with PBS containing 5 mM MgCl₂ and 5 mM CaCl₂ and luciferase activity was assayed using a stabilized luciferin substrate (SteadyLite, PerkinElmer). Emitted light was measured using a luminometer (Envision plate reader, PerkinElmer) and expressed as relative light units. FIG. 7 shows that the greatest synergy between chitosan-arginine (18 kD, 25% functionalization) and Lipofectamine 2000 is observed at ratios of DNA:chitosan-arginine (18 kD, 25% functionalization) of 1:5 and 1:10. Expression of luciferase with Lipofectamine plus DNA:chitosan-arginine (18 kD, 25% functionalization) of 1:5 is 10 fold greater than Lipofectamine alone, and 8,000 fold greater than chitosan-arginine (18 kD, 25% functionalization) of 1:5 alone. Expression of luciferase with Lipofectamine plus DNA:chitosan-arginine (18 kD, 25% functionalization) of 1:10 is also 10 fold greater than Lipofectamine alone, and 85 fold greater than chitosan-arginine (18 kD, 25% functionalization) of 1:10 alone.

### Example 8

### Luciferase Transfection into 293T, A549, Caco2, A431 and 3T3 Cells

4×10⁴ HEK293T, 3×10⁴NIH3T3, 3×10⁴ A549, 1×10⁴ Caco2, or 2×10⁴ A431 cells in 100 ml of DMEM (all amounts and volumes are given on a per well basis) were seeded into 96 cell plates one day before transfection. Immediately before transfection the medium was replaced with 100 μl/well DMEM supplemented with 10% fetal bovine serum without antibiotics. 0.2 μg/well of a luciferase plasmid with CMV promoter (pGL4.51, Promega) were diluted into DMEM medium (pH7.4). Chitosan-arginine (18 kD, 25% functionalization) was added to give DNA mass/Chitosan derivative mass ratio of 1:5, 1:25, or 1:50. Lipofectamine 2000 (Invitrogen Cat#116680027) was used as the positive control and added to relevant tubes to test luciferase activity with DNA plus both transfection reagent and chitosan derivative. Lipofectamine 2000 was used at DNA:Lipofectamine 2000 mass:volume ratio of 1:2.5. All total volumes of mixtures were adjusted to be the same before transfection. Mixtures were incubated at room temperature for 20 minutes, then added gently to triplicate wells of cells in 96 well plates. Cells were incubated at 37° C. in a CO₂ incubator for 24 hours prior to testing for transgene expression. After incubation, medium was replaced with PBS containing 5 mM MgCl₂ and 5 mM CaCl₂ and luciferase activity was assayed using a stabilized luciferin substrate (SteadyLite, PerkinElmer). Emitted light was measured using a luminometer (Envision plate reader, Perkin Elmer) and expressed as relative light units.

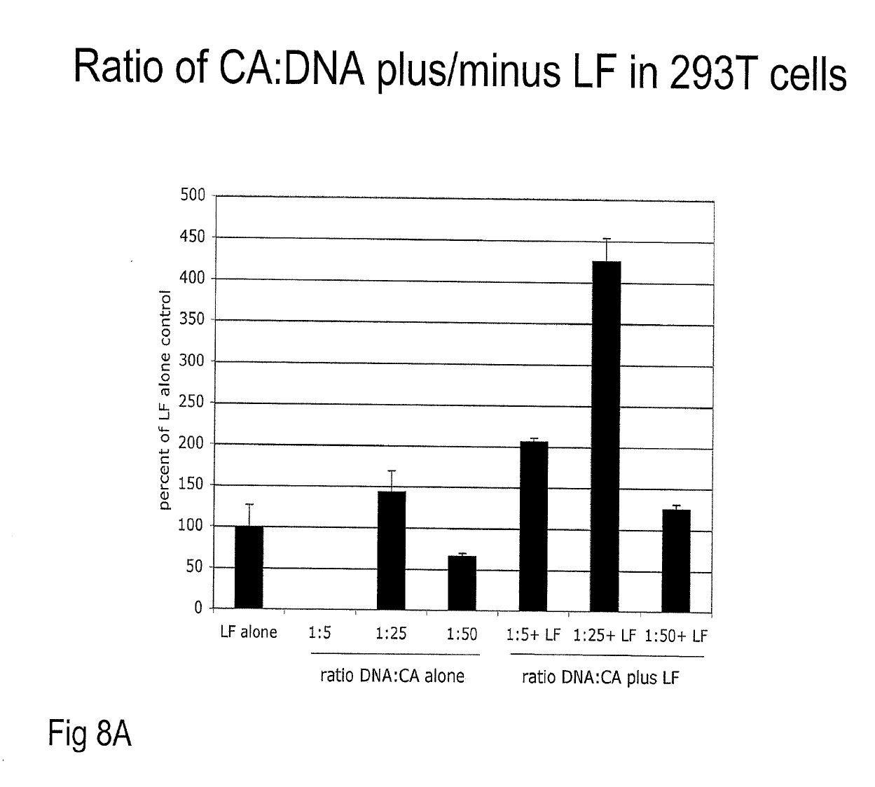

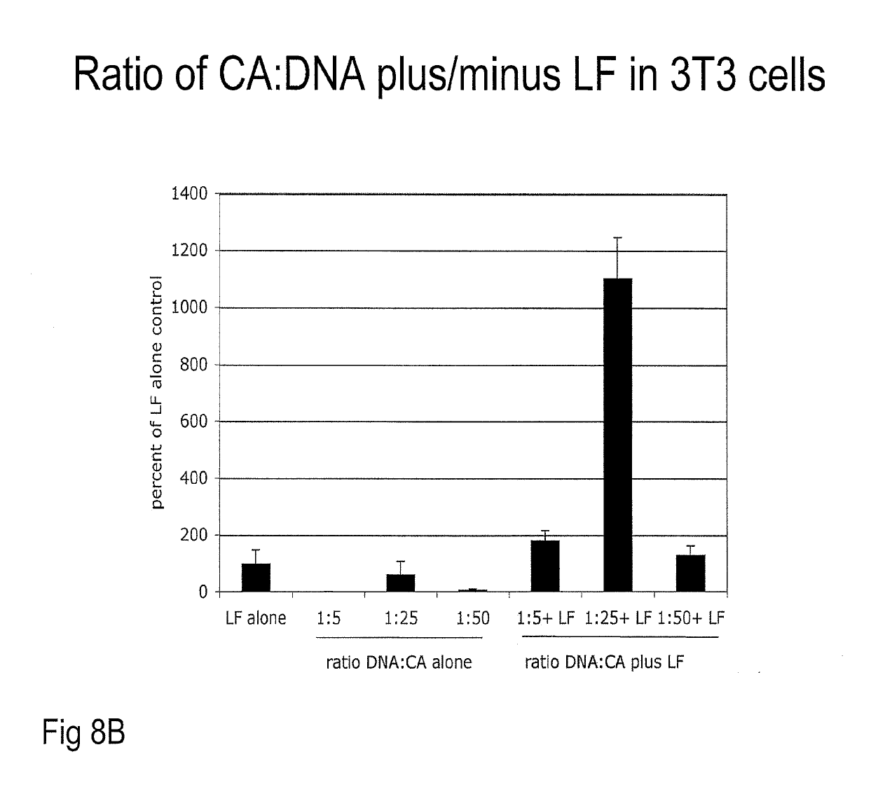

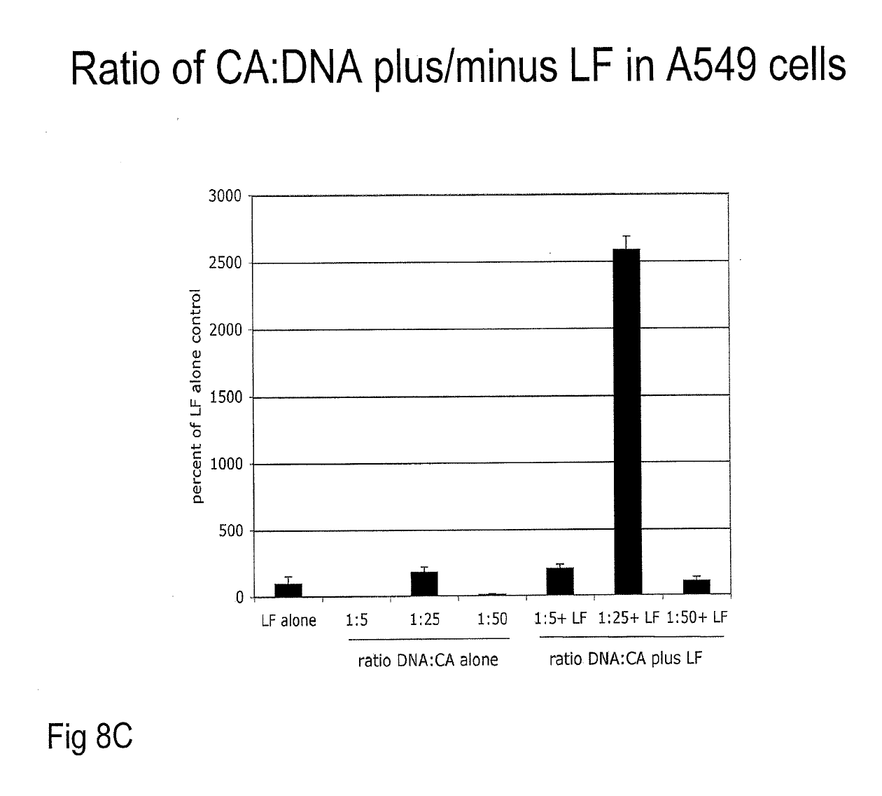

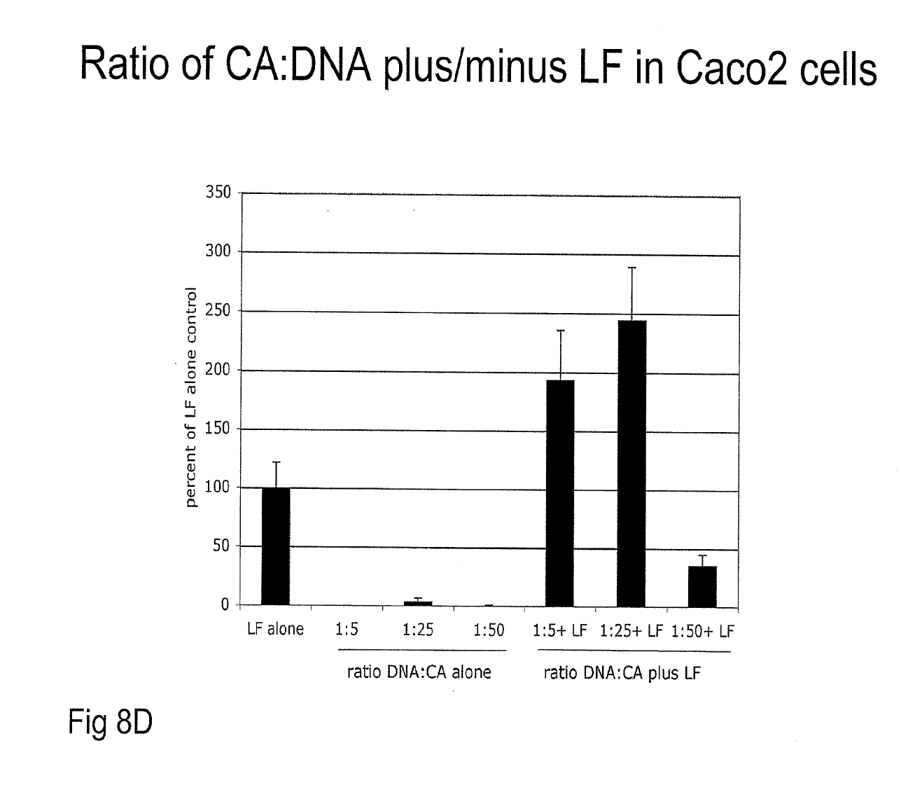

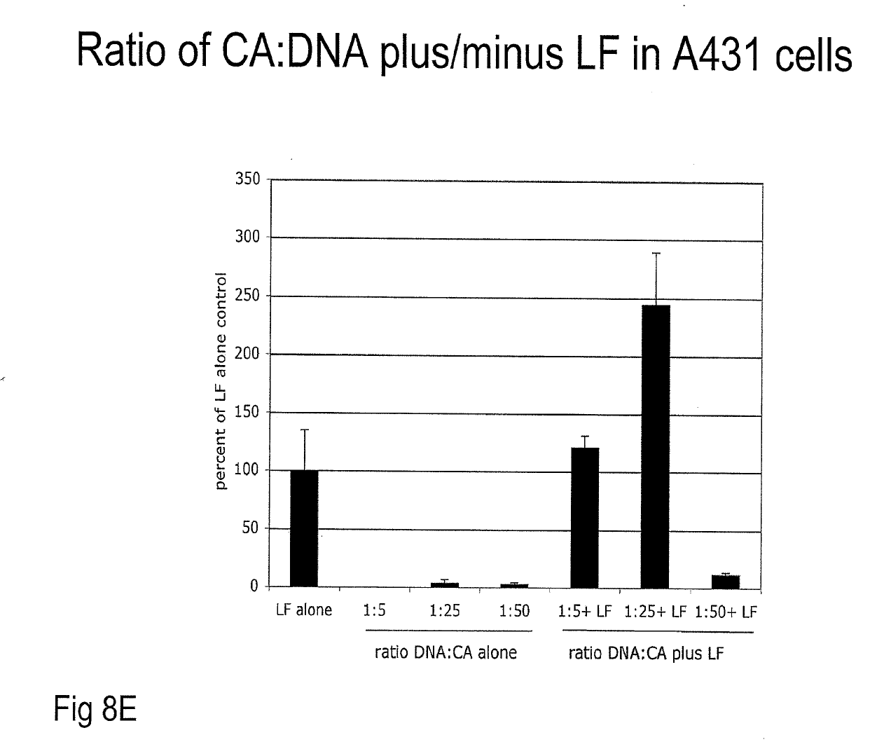

Relative luciferase activities (percentage of Lipofectamine 2000 only control) for each cell line are shown in FIGS. 8A-8E.

### Example 9

### The sensitization of Caco2 and A549 Cells by Chitosan Derivatives

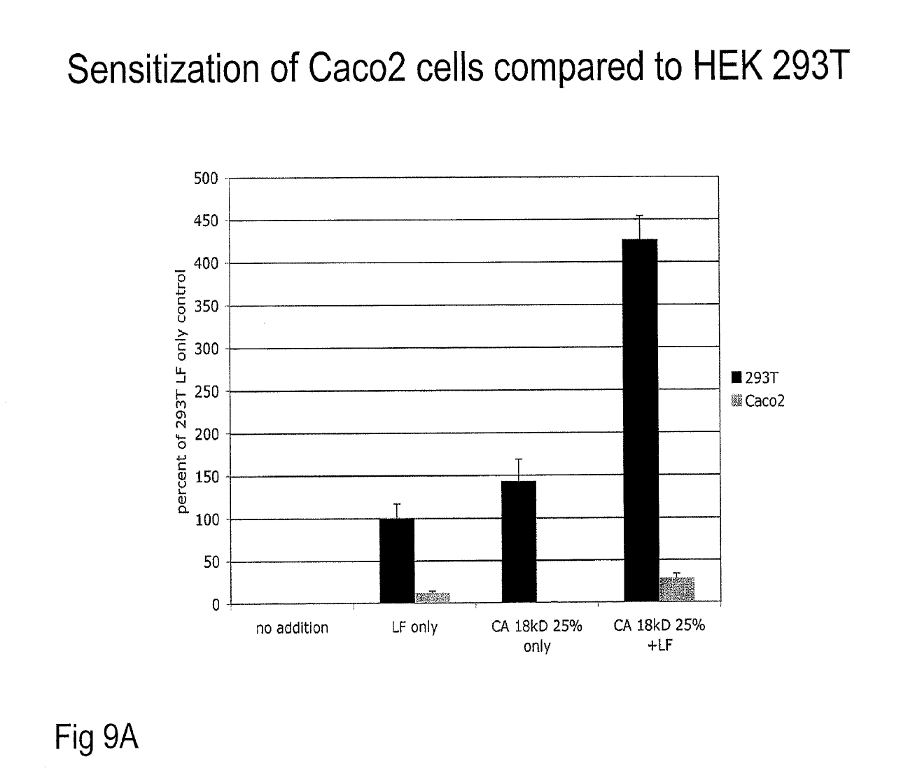

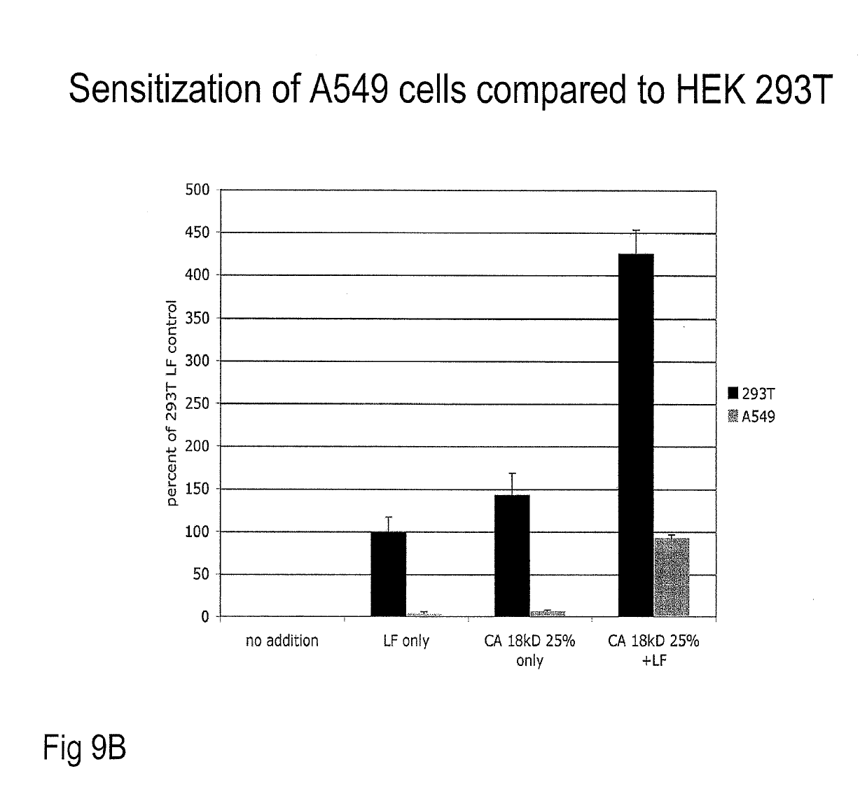

Caco2 and A549 cells were transfected as described in Example 8. Chitosan-arginine (18K, 25% functionalization) was tested. The sensitization of hard-to-transfect Caco2 and A549 cells by the addition of chitosan derivatives to lipofectamine is shown in FIGS. 9A and 9B.

### Example 10

### Increased Transfection Ability into Adipose Derived Stem Cells

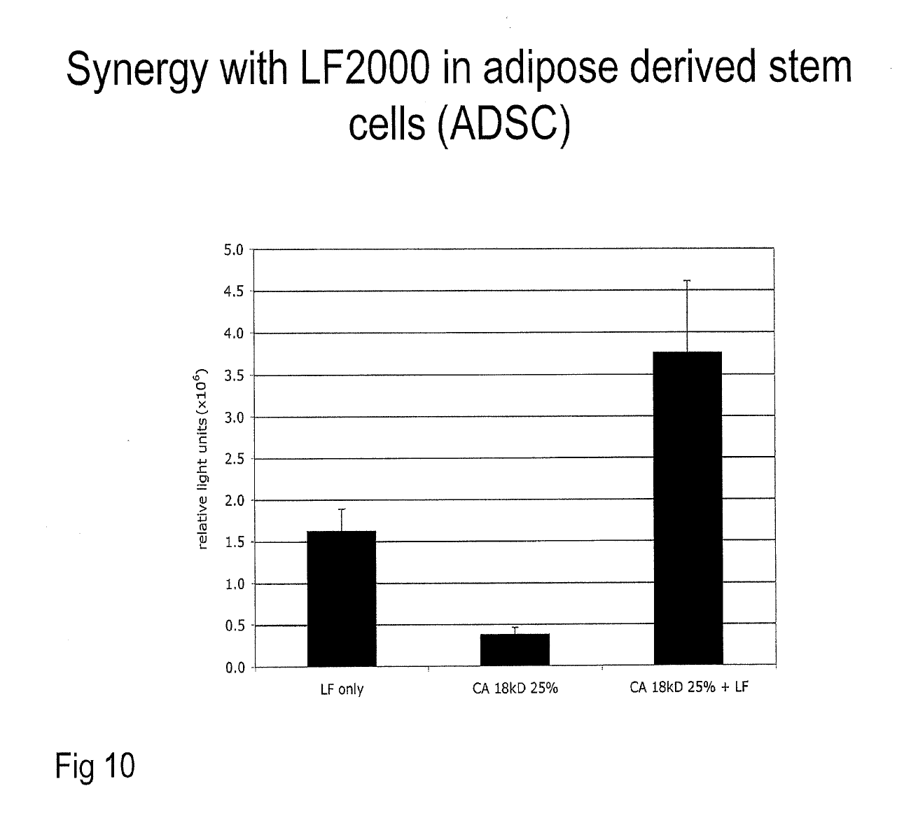

2×10³ Adipose derived stem cells (ADSC) in 100 μl of complete Mesenpro medium (all amounts and volumes are given on a per well basis) were seeded into 96 cell plates one day before transfection. Immediately before transfection medium was replaced with complete Mesenpro medium containing serum but without any antibiotics 0.2 μg/well of a luciferase plasmid with CMV promoter (pGL4.51, Promega) were diluted into Mesenpro basal medium (pH7.4). Chitosan-arginine (18 kD, 25% functionalization) was added to give DNA mass/Chitosan derivative mass ratio of 1 to 25. Lipofectamine 2000 was used as the positive control and was added to relevant tubes to test luciferase activity with DNA plus both transfection reagent and chitosan derivative. Lipofectamine 2000 was used at DNA:Lipofectamine 2000 mass:volume ratio of 1:2.5. All total volumes of mixtures were adjusted to be the same before transfection. Mixtures were incubated at room temperature for 20 minutes, then added gently to triplicate wells of cells in 96 well. Cells were incubated at 37° C. in a CO₂ incubator for 24 hours prior to testing for transgene expression. After incubation, medium was replaced with PBS containing 5 mM MgCl₂ and 5 mM CaCl₂ and luciferase activity was assayed using a stabilized luciferin substrate (SteadyLite, PerkinElmer). Emitted light was measured using a luminometer (Envision plate reader, Perkin Elmer) and expressed as relative light units. FIG. 10 shows that transfection with both chitosan-arginine (18 kD, 25% functionalization) and Lipofectamine 2000 is 2.4 fold greater than Lipofectamine 2000 alone, and 10 fold greater than chitosan-arginine (18 kD, 25% functionalization) alone.

### Example 11

### Chitosan-Arginine as a Transfection Reagent for Suspension cultured CHO-K1 Cells

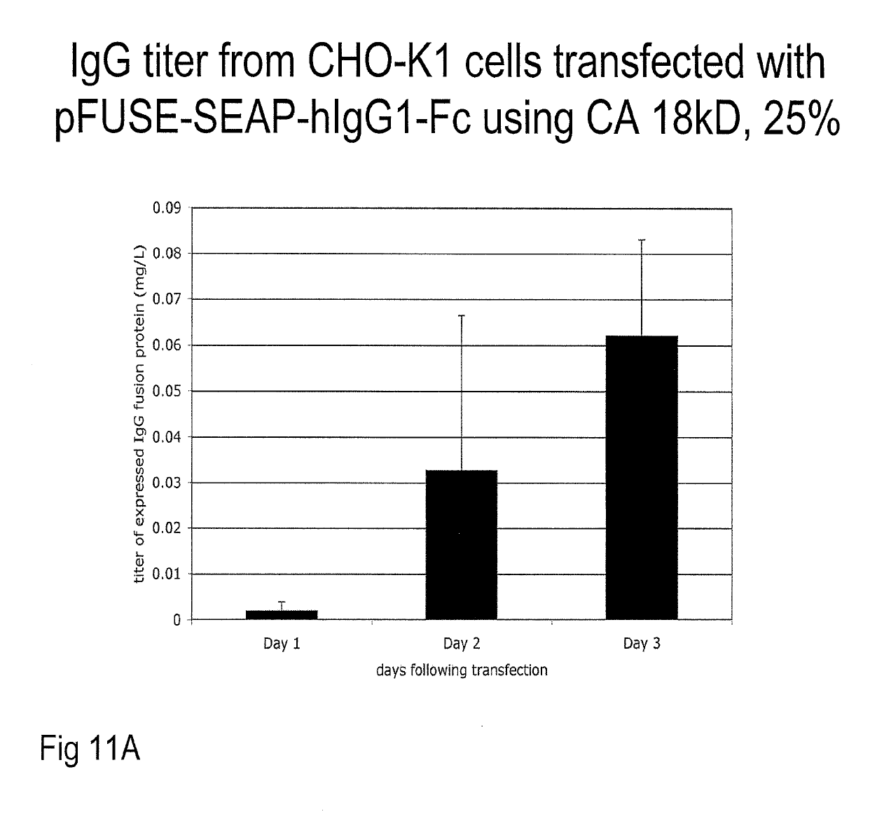

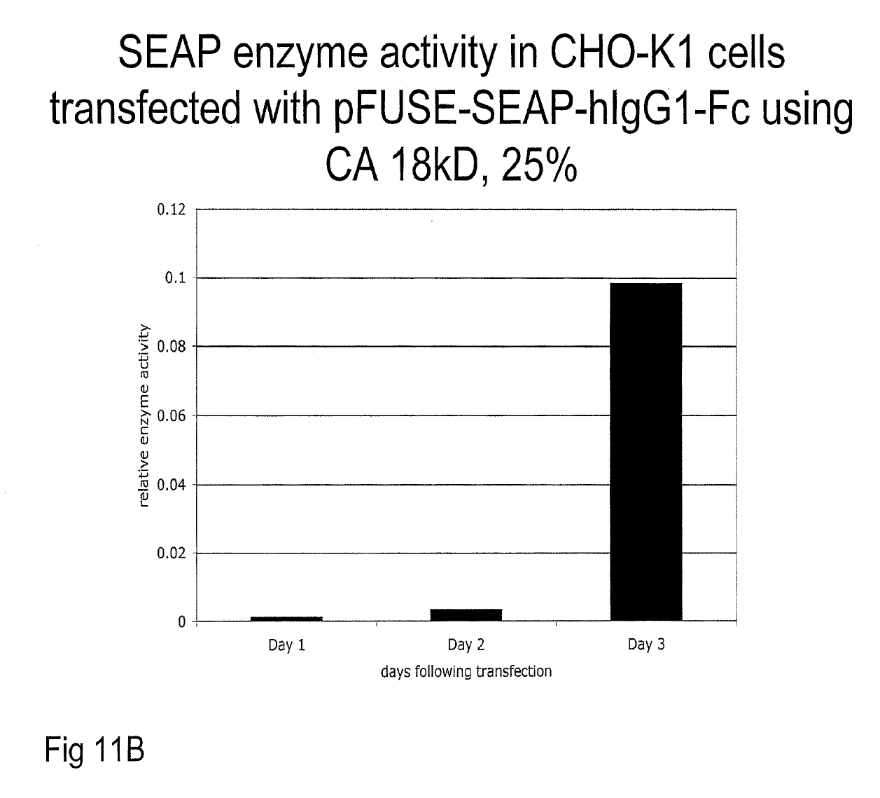

6×10⁷ CHO-K1 suspension adapted cells were transfected with pFUSE-SEAP-hIgG1-Fc using 150 μg DNA and 3750 μg chitosan-arginine (18 kD, 25% functionalization) (DNA: chitosan-arginine (18 kD, 25% functionalization) ratio=1:25) in a total volume of 3 ml of ProCHO5 medium (Lonza) supplemented with L-glutamine, hypoxanthine, thymidine and F-68. DNA was added to the cells first independently of the CA. Subsequently, the CA was added to the cell and DNA mixture. Cell suspension was aliquoted into three wells of a 6-well plate (35 mm wells) and incubated for 3 hours at 37° C. on a rocker set to 110 rpm. After this initial incubation cells were diluted by addition of 4 ml of fresh medium containing valporic acid and incubated at 33° C. shaking at 110 rpm. One well of cells was taken for analysis at 24, 48 and 96 hours post transfection. Amount of transfection was measured by assaying the amount of IgG-SEAP fusion protein present in the culture medium using both an ELISA assay (FIG. 11A) and enzyme activity assay (FIG. 11B). Chitosan-arginine (18 kD, 25% functionalization) is able to transfect CHO-K1 cells with the amount of expressed reporter gene increasing over the three days of incubation following transfection.

What is claimed is:

1.-195. (canceled)

196. A method of transfecting a cell with a nucleic acid, the method comprising:
providing a cell; and
contacting said cell with a composition comprising the nucleic acid, and a functionalized chitosan of the following formula (I): 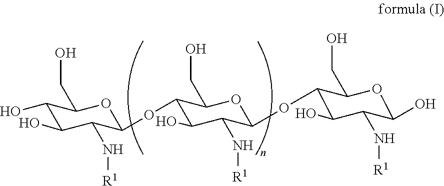
wherein:
n is an integer between 20 and 6000; and
each R¹ is independently selected for each occurrence from hydrogen, acetyl, and a group of formula (II): wherein, formula (II) is selected from 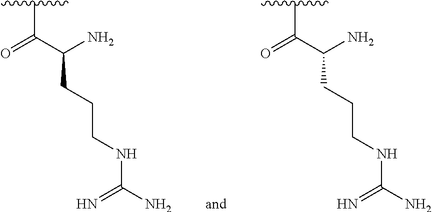
wherein at least 25% of R¹ substituents are H, at least 1% of R¹ substituents are acetyl, and at least 2% of R¹ substituents are a group of formula (II).

197. The method of claim 196, wherein the composition comprises a complex or particle, wherein the complex or particle comprises the chitosan derivative and the nucleic acid.

198. The method of claim 196, wherein 55-90% of R¹ substituents are hydrogen, 4-20% of R¹ substituents are acetyl, 4-30% of R¹ substituents are a group of formula (II).

199.-200. (canceled)

201. The method of claim 196, wherein the molecular weight of the functionalized chitosan is between about 10,000 and about 1,000,000 Da.

202. The method of claim 196, wherein the functionalized chitosan is soluble in aqueous solution between pH 6 and pH 8.

203. The method of claim 196, wherein the functionalized chitosan is substantially free of other impurities.

204. The method of claim 196, wherein the composition further comprises a lipid or a lipid formulation.

205. The method of claim 196, wherein the nucleic acid comprises a DNA or RNA.

206. The method of claim 196, wherein the nucleic acid comprises a therapeutic gene.

207. The method of claim 196, wherein the nucleic acid comprises a vector.

208. A kit comprising a functionalized chitosan of formula (I): 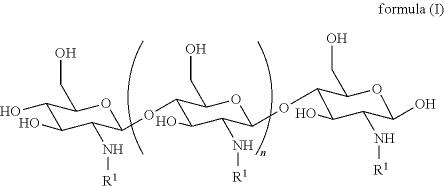
wherein:
n is an integer between 20 and 6000; and
each R¹ is independently selected for each occurrence from hydrogen, acetyl, and
a group of formula (II): wherein, formula (II) is selected from 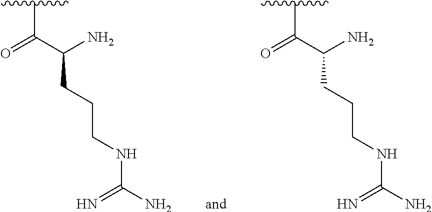
wherein at least 25% of R¹ substituents are H, at least 1% of R¹ substituents are acetyl, and at least 2% of R¹ substituents are a group of formula (II); and
instructions for use to transfect a nucleic acid to a cell.

209. The kit of claim 208, further comprising a nucleic acid.

210. A pharmaceutical composition comprising a nucleic acid and a functionalized chitosan of formula (I): 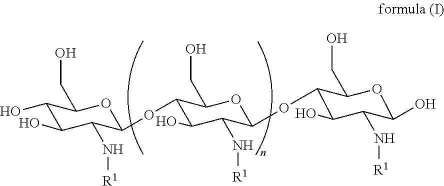
wherein:
n is an integer between 20 and 6000; and
each R¹ is independently selected for each occurrence from hydrogen, acetyl, and
a group of formula (II): wherein, formula (II) is selected from 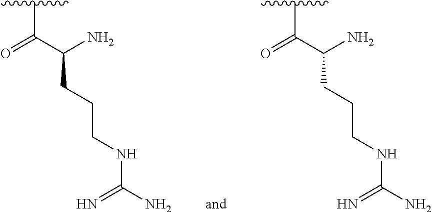
wherein at least 25% of R¹ substituents are H, at least 1% of R¹ substituents are acetyl, and at least 2% of R¹ substituents are a group of formula (II).

211. The composition of claim 210, comprising a complex or particle, wherein the complex or particle comprises the chitosan derivative and the nucleic acid.

212. The composition of claim 210, further comprising a second transfection reagent.

213. The composition of claim 210, wherein the nucleic acid comprises a DNA or RNA.

214. The composition of claim 210, wherein the nucleic acid comprises a therapeutic gene.

215. A reaction mixture comprising a nucleic acid and a functionalized chitosan of formula (I): 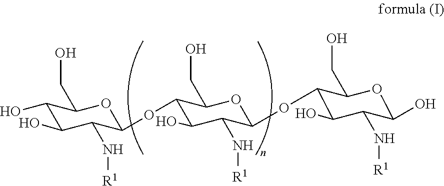
wherein:
n is an integer between 20 and 6000; and
each R¹ is independently selected for each occurrence from hydrogen, acetyl, and
a group of formula (II): wherein, formula (II) is selected from 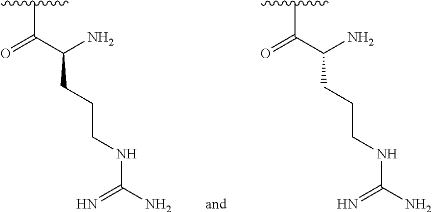
wherein at least 25% of R¹ substituents are H, at least 1% of R¹ substituents are acetyl, and at least 2% of R¹ substituents are a group of formula (II).

216. The reaction mixture of claim 215, wherein the nucleic acid comprises a DNA or RNA.

217.-219. (canceled)

220. A chitosan derivative/nucleic acid complex comprising:
a functionalized chitosan of formula (I): 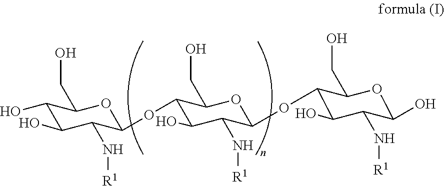
wherein:
n is an integer between 20 and 6000; and
each R¹ is independently selected for each occurrence from hydrogen, acetyl, and
a group of formula (II): wherein, formula (II) is selected from 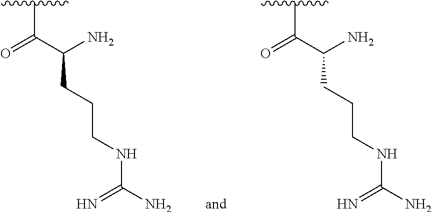
wherein at least 25% of R¹ substituents are H, at least 1% of R¹ substituents are acetyl, and at least 2% of R¹ substituents are a group of formula (II) and
a nucleic acid.

221.-223. (canceled)

224. A method of delivering a nucleic acid to a cell, the method comprising:
providing chitosan derivative/nucleic acid complex comprising a functionalized chitosan of formula (I): 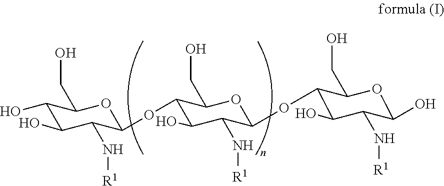
wherein:
n is an integer between 20 and 6000; and
each R¹ is independently selected for each occurrence from hydrogen, acetyl, and
a) a group of formula (II): wherein, formula (II) is selected from 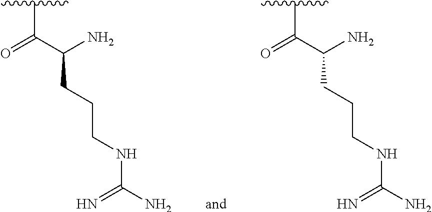
wherein at least 25% of R¹ substituents are H, at least 1% of R¹ substituents are acetyl, and at least 2% of R¹ substituents are a group of formula (II).

225. A method of transfecting a cell with a nucleic acid, the method comprising:
providing a cell;
contacting the cell with a functionalized chitosan of formula (I): 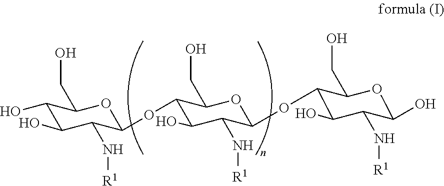
wherein:
n is an integer between 20 and 6000; and
each R¹ is independently selected for each occurrence from hydrogen, acetyl, and
a group of formula (II): wherein, formula (II) is selected from 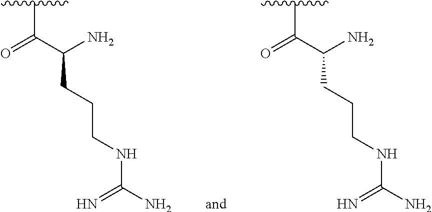
wherein at least 25% of R¹ substituents are H, at least 1% of R¹ substituents are acetyl, and at least 2% of R¹ substituents are a group of formula (II); and contacting the cell with a nucleic acid, thereby transfecting a nucleic acid to a cell.
